(19) United States
(12) Patent Application Publication
(10) Pub. No.: US 2002/0086427 A1
(43) Pub. Date: Jul. 4, 2002

(54) Inducible eukaryotic expression system that regulates protein translation

(76) Inventors: Jeffrey M. Leiden, Glencoe, IL (US); Deborah Marshall, Brookline, MA (US)

Correspondence Address: LAHIVE & COCKFIELD, 28 STATE STREET, BOSTON, MA, 02109, US

(21) Appl. No.: 09/815,980

(22) Filed: Mar. 22, 2001

Related U.S. Application Data
(60) Provisional application No. 60/191,568, filed on Mar. 23, 2000.

(51) Int. Cl. A61K 31/70; C12N 15/63

(52) U.S. Cl. 435/455

(57) ABSTRACT

A system for regulating the expression of a gene in a eukaryotic cell is provided, in which the expression of a desired gene can be activated or deactivated according to deliberate intentions (i.e., via an inducible signal) and in which regulation of gene expression occurs at the level of translation of the gene. This regulation is accomplished by, first, the introduction of at least one mutation into the coding sequence of the gene of interest. This mutation(s) causes a decrease or alteration of translation, and, hence, a decrease or alteration of expression of the desired gene. The method of the invention further involves contacting the eukaryotic cell containing the now mutated gene of interest with an agent that is able to suppress the effect of the mutation, thus allowing translation, and, hence, expression of the desired gene. Preferably, the method involves introduction of a stop codon mutation, which is suppressed by an aminoglycoside. Nucleic acid compositions for use in the system of the invention, and kits for carrying out the methods of the invention, are also provided.

### RELATED APPLICATIONS

This application claims the benefit of priority under 35 U.S.C. 119(e) to co-pending U.S. Provisional Application No. 60/191,568, filed Mar. 23, 2000, the entire contents of which are hereby incorporated by reference.

### BACKGROUND OF THE INVENTION

Current biotechnology affords the direct delivery of polypeptides by inserting an appropriate heterologous gene (“transgene”) into a host cell or organism and subsequently allowing the host to produce the polypeptide. In broad terms, these techniques involve the incorporation of the transgene into an expression vector that is capable of stable existence in host cells and that supports expression of the transgene. More recently, the development of expression vectors that incorporate regulatory transcription elements that allow for inducible expression of the transgene have been the subject of considerable effort. Generally, such inducible expression vectors are designed to be controlled by a deliberate external stimulus. Ideally, such a system would not only mediate an “on/off” status for gene expression but would also permit limited expression of a gene at a defined level (“fine tuning”). An expression system that can be reliably modulated from without would be very useful in a wide range of research and therapeutic efforts. For example, functional analysis of cellular proteins is greatly facilitated by the ability to effect changes in the expression level of the corresponding gene, allowing subsequent analysis of the accompanying phenotype. Furthermore, many genetic disorders manifest themselves in corresponding polypeptides, which are mutated or aberrantly expressed. Genetic therapy using such an inducible expression vector can be used to treat such diseases through measured delivery of a desired polypeptide.

Attempts to deliberately control genetic expression via an external stimulus have been made using various inducible eukaryotic promoters, such as those responsive to heavy metal ions (Mayo et al, 1982, Cell, 29:99-108; Brinster et al, 1982, Nature, 296:39-42; Searle et al, 1985, Mol. Cell. Biol., 5:1480-1489), heat shock (Nouer et al,1991, in Heat Shock Response, e.d. Nouer, L., CRC, Boca Raton Fla., pp167-220) or hormones (Lee et al,l981, Nature 294:228-232; Hynes et al, 1981, Proc. Natl. Acad. Sci. USA 78:2038-2042; Klock et al, 1987, Nature 329:734-736; Israel & Kaufman, 1989, Nucl. Acids Res. 17:2589-2604), tetracycline (Gossen and Bujard, 1992, Proc. Natl. Acad. Sci. USA 89:5547-5551), and rapamycin (Ye et al, 1999, Science, 293:88-91). However, these systems generally suffer one or more of several shortcomings. For example, the inducer component (e.g. heavy metal ions, heat shock proteins, steroid hormones, or prokaryotic proteins) often evokes pleiotropic effects, including possible immunogenicity, which can complicate analyses and diminish usefulness. In addition, many promoter systems exhibit high levels of basal activity (“leakiness”) in the non-induced state, which leads to lowered induction factors. Furthermore, the tetracycline- and rapamycin-based systems mentioned here have an additional cumbersome feature in that accessory transgenes must be co-introduced and simultaneously expressed with the transgene of interest within a single cell. These accessory transgenes are potentially immunogenic, thereby increasing the risk of inflammation and elimination of transgene expression.

Therefore, there still exists a need to develop an inducible eukaryotic expression system that is simple (e.g., the gene of interest is the only gene expressed), efficient and effective.

### SUMMARY OF THE INVENTION

This invention provides a system for regulating the expression of a gene in a eukaryotic cell in which the expression of a desired gene can be activated or deactivated according to deliberate intentions (i.e., via an inducible signal) and in which regulation of gene expression occurs at the level of translation of the gene. This regulation is accomplished by, first, the introduction of at least one mutation into the coding sequence of the gene of interest. This mutation(s) causes a decrease (i.e., inhibition) or alteration of translation, and, hence, a decrease (i.e., inhibition) or alteration of expression of the desired gene. The method of the invention further involves contacting the eukaryotic cell containing the now mutated gene of interest with an agent that is able to suppress the effect of the mutation, thus allowing translation, and, hence, expression of the desired gene.

In a preferred embodiment of the invention, the mutation(s) that is introduced into the coding region of a desired gene is a mutation(s) that creates a stop codon, thereby causing premature termination of translation of the gene. In this embodiment, the agent that is used to suppress the mutation(s) is an aminoglycoside, which restores translation of the gene by allowing for “read through” of the stop codon mutation(s). Examples of preferred aminoglycosides for use in this embodiment of the invention include Hygromycin-B, Gentamycin, Paromomycin, Tobramycin, Lividomycin and G418.

In another embodiment, the mutation(s) that is introduced into the coding region of a desired gene is a mutation(s) that creates a missense mutation, thereby causing mistranslation of the gene. In this embodiment, the agent that is used to suppress the mutation(s) is a suppressor tRNA molecule, which restores translation of the gene by allowing for proper translation of the gene.

In yet another embodiment, the mutation(s) that is introduced into the coding region of a desired gene is a mutation(s) that creates a ribozyme cleavage site, leading to digestion of the mRNA In this embodiment, the agent that is used to suppress the mutation(s) is an inhibitor of the ribozyme.

The invention allows for inducible expression of a gene in eukaryotic cells for any purpose, including for therapeutic purposes. The desired gene to be regulated can be any gene, provided that the appropriate mutation is introduced in accordance with the invention. In various embodiments, the desired gene that is used in the method of the invention is chosen from the group consisting of erythropoietin, insulin, a vascular endothelial cell growth factor (VEGF), a fibroblast growth factor (FGF), hypoxia- inducible factor-1α (HIF-1α), Factor VIII, Factor IX, growth hormone, anti-angiogenesis polypeptides (such as endostatin or angiostatin) and herpes simplex virus thymidine kinase.

Typically, the desired gene is incorporated into a eukaryotic expression vector prior to being provided to (e.g., introduced into) the eukaryotic cell. Also typically, the eukaryotic expression vector contains genetic elements that support the expression of the incorporated desired gene. Some or all of these elements may be chosen from the group consisting of enhancers, promoters, and polyadenylation signal-sequences. Examples of suitable regulatory elements include EF-1α promoter, 4F2 enhancer, adevovirus enhancer, adenovirus promoter, CMV enhancer, CMV promoter, RSV enhancer, RSV promoter, bovine growth hormone polyadenylation signal-sequence and SV40 polyadenylation signal-sequence. In certain embodiments, the eukaryotic expression vector is, for example, a retroviral vector, a lentiviral vector, an adenoviral vector or an adeno-associated viral vector.

The invention also provides that the desired gene can be delivered into the eukaryotic cell or into a subject. Nucleic acids of the invention may be delivered into cells by one or more methods for introducing nucleic acid into cells, such as transfection, lipofection, calcium phosphate precipitation, electroporation, microprojectile bombardment, viral infection (e.g., retroviral, lentiviral or adenoviral infection). In a further embodiment, the invention provides for the delivery of a desired gene to a subject (e.g., a patient) by supplying cultured cells to the subject after the desired mutated gene has been introduced into the cultured cells. Cultured eukaryotic cells that can be used for any of the embodiments of the invention include hematopoietic stem cells, myoblasts, hepatocytes, lymphocytes, skin epithelium, cardiomyocytes, vascular smooth muscle cells, endothelial cells, neurons and airway epithelium. Preferably, the cultured cells are from the same subject into which they will be reintroduced following introduction of the desired mutated gene into the cells. In an alternative embodiment, the desired mutated gene is provided to the subject by directly delivering the gene into cells of the subject. Types of cells into which the desired mutated gene can be directly delivered in a subject include hematopoietic stem cells, myoblasts, hepatocytes, lymphocytes, skin epithelium, cardiomyocytes, vascular smooth muscle cells, endothelial cells, neurons and airway epithelium. Once the desired mutated gene is present in the subject, expression of the gene is regulated by modulation, in the subject, of the agent that suppresses the mutation introduced into the desired gene (e.g., by administration of an aminoglycoside to the subject).

Another aspect of the invention pertains to particular isolated nucleic acid compositions corresponding to genes of interest containing one or more stop codon mutations in the coding region of the gene. Such nucleic acid compositions can be used in the regulatory system of the invention. Examples include: an isolated human erythropoietin gene comprising at least one stop codon mutation within the coding region of the gene; an isolated human insulin gene comprising at least one stop codon mutation within the coding region of the gene; an isolated human VEGF gene comprising at least one stop codon mutation within the coding region of the gene; an isolated human FGF gene comprising at least one stop codon mutation within the coding region of the gene; an isolated human HIF-1α gene comprising at least one stop codon mutation within the coding region of the gene; an isolated human Factor VIII gene comprising at least one stop codon mutation within the coding region of the gene; an isolated human Factor IX gene comprising at least one stop codon mutation within the coding region of the gene; an isolated human growth hormone gene comprising at least one stop codon mutation within the coding region of the gene; an isolated human endostatin gene comprising at least one stop codon mutation within the coding region of the gene; an isolated human angiostatin gene comprising at least one stop codon mutation within the coding region of the gene; and an isolated herpes simplex virus thymidine kinase gene comprising at least one stop codon mutation within the coding region of the gene.

Kits for carrying out the methods of the invention are also encompassed by the invention. Such kits can contain, for example, an isolated gene comprising at least one stop codon mutation within a coding region of the gene and an aminoglycoside for suppression of the at least one stop codon mutation in the gene. In a preferred embodiment, the gene is a human erythropoietin gene and the aminoglycoside is Gentamycin.

### BRIEF DESCRIPTION OF THE DRAWINGS

FIG. 1 is a bar graph depicting the results of a transfected cell experiment assaying the ability of different aminoglycosides to permit translation of a human Epo (hEpo) gene into which a stop codon mutation has been introduced within the coding region of the gene. Three different mutated human Epo genes were assayed, representing three different introduced stop codon mutations. Results are shown for 24, 48 and 72 hours of treatment with two different aminoglycosides, G418 and gentamycin sulfate (GS). For each of the three stop codon mutations, fold-induction of expression was calculated based on comparison with hEpo production in cells from the same transfected culture that were not treated with aminoglycoside.

FIG. 2 is a bar graph depicting the results of additional transfected cell experiments assaying the ability of aminoglycosides to permit translation of eight different mutated hEpo genes into which a stop codon mutation had been introduced within the coding region of the gene. Results are shown for 24 and 48 hours of treatment with either G418 or gentamycin sulfate (GS). For certain constructs (pVR1012hEPO.307, .434, .208, .214 and .199) results are shown for two different clones.

FIG. 3 is a bar graph depicting the results of experiments assaying the biological activity of hEpo expressed upon aminoglycoside (G418) treatment of cells transfected with different mutated hEpo genes into which a stop codon mutation had been introduced within a coding region of the gene. The biological activity of hEpo was assessed on Epo-dependent UT7 cells.

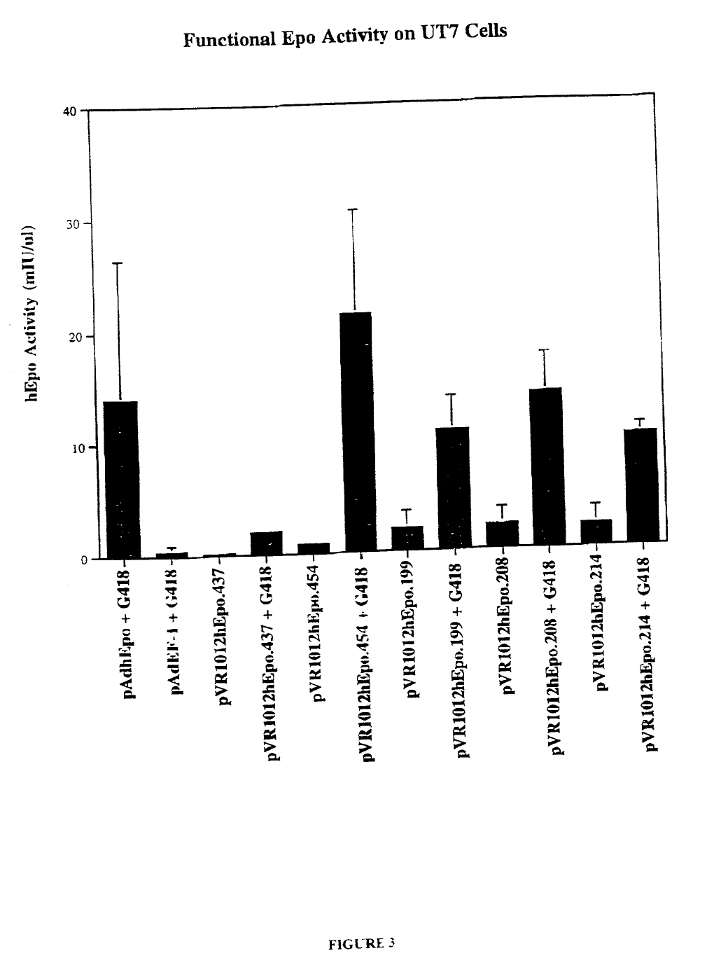

### DETAILED DESCRIPTION OF THE INVENTION

The invention described herein pertains to methods of regulating the expression of a gene in a eukaryotic cell, and compositions for use in such methods. The methods of the invention involve the introduction of at least one mutation into a coding region of a gene, such that translation of the coding region is terminated or altered at the codon corresponding to the mutation. Thus, as a result of this mutation, a functional gene product encoded by the gene is not produced in the eukaryotic cell. The method of the invention further involves contacting the eukaryotic cell with an agent that suppresses the mutation in the coding region of the gene, such that a functional gene product is now produced by the eukaryotic cell. The ability to modulate the contact of the eukaryotic cell with this agent allows for regulated expression of the gene of interest in accordance with the invention described herein. In view of the forgoing, the invention broadly provides methods and compositions for modulating the expression of a gene in a eukaryotic cell.

So that the invention may be more readily understood, a number of terms are first defined.

As used herein, the term “expression” refers to the fundamental biological process of producing an active polypeptide from the nucleic acid molecule or gene that encodes it. The term, as used herein, is intended to encompass some or all of the steps of normal genetic expression including, transcriptional initiation, transcription, transcript processing, translation, and post-translational processing.

As used herein, the term “regulating expression” refers generally to the quality of being able to modulate the expression of a gene, either positively (i.e. increasing its expression) or negatively (i.e. diminishing or halting its expression). It is furthermore intended to include modulation which occurs through deliberate design, i.e. through the use of the invention described herein.

As used herein, the gene to be regulated using the system of the invention is referred to interchangeably as a “gene”, a “desired gene” or a “gene of interest.” In one embodiment, the desired gene is a “eukaryotic gene”, which is intended to refer to a gene of a eukaryotic cell or organism, or a gene derived from such a cell or organism (e.g., the gene may be modified in its nucleotide sequence as compared to a wildtype gene from a eukaryotic cell or organism but is based on the nucleotide sequence from a eukaryotic cell or organism). The term “eukaryotic gene” does not include prokaryotic genes, such as the bacterial chloramphenicol acetyltransferase (CAT) gene. In another embodiment, the desired gene is a “gene of therapeutic interest”, which is intended to encompass genes whose expression can ameliorate a disease condition or disorder. A gene of therapeutic interest may act by supplying a gene product of therapeutic benefit (e.g., replacement of a defective gene product in a cell or supplementing a defective gene product in a cell) or by inhibiting an undesireable process or activity, such as inhibiting the growth or spread of cancer cells. In another embodiment, a desired gene is a “viral gene”, which is intended to refer to a gene of a virus, or a gene derived from a virus (e.g., the gene may be modified in its nucleotide sequence as compared to a wildtype gene from a virus but is based on the nucleotide sequence from a virus).

In certain embodiments of the invention, the desired gene can be any gene with the proviso that it is not a bacterial chloramphenicol acetyltransferase (CAT) gene. Burke J.F. and Mogg, A. E. (Nucl. Acids Res. (1985) 13:6265-6272) and Martin, R. et al. (Mol. Gen. Genet. (1989) 217:411-418) report the suppression, using an aminoglycoside, of a nonsense mutation in a bacterial chloramphenicol acetyl transferase gene introduced into mammalian cells. However, the mutated bacterial CAT gene was used only to test the effect of aminoglycosides in mammalian cells and the authors did not teach or suggest the deliberate introduction of stop codon mutations into genes for regulatory purposes, which deliberately introduced mutations then could be suppressed by administration of aminoglycosides.

In certain embodiments of the invention, the desired gene can be any gene with the proviso that it is not a cystic fibrosis transmembrane conductance regulator (CFTR) or a dystrophin gene. Howard, M. et al. (Nature Medicine (1996) 2:467-469) and Bedwell, D.M et al. (Nature Medicine (1997) 3:1280-1284) report the suppression of naturally-occurring premature stop codon mutations in the CFTR gene using aminoglycosides, while Barton-Davis, E.R. et al. (J. Clin. Invest. (1999) 104:375-381) report the suppression of naturally-occurring premature stop codon mutations in the dystrophin gene using aminoglycosides. In each of these reports, however, aminoglycosides were used to suppress naturally-occurring stop codon mutations to restore normal function of the gene; the authors did not teach or suggest the deliberate introduction of stop codon mutations into genes for regulatory purposes, which deliberately introduced mutations then could be suppressed by administration of aminoglycosides.

As used herein, the term “introducing at least one mutation into a transcribed region of a gene” is intended to refer to the deliberate modification of a transcribed region of the gene and is not intended to encompass naturally occurring mutations that may exist in the gene.

As used herein, the term “coding region” refers to the nucleotide sequence of a gene which specifies the codons for translation into polypeptides, e.g. the region that codes for the amino acids. Coding region sequences are to be distinguished from other non-coding sequences associated with genes, e.g. 5′ untranslated regions, 3′ untranslated regions, and introns.

As used herein, the term “terminates” (as in “translation of the gene terminates”) is intended to encompass both partial and complete termination of translation of the gene at one or more introduced stop codon mutations.

As used herein the term “is altered” (as in “the translation of the gene is altered” is intended to refer to a change in the translation of the gene such that a gene product other than that encoded by the wildtype version of the gene is produced, or no gene product is produced, or a decreased amount of gene product is produced.

As used herein, the term “a functional gene product of the gene is not produced in the cell” is intended to encompass situations in which no gene product is made in the cell, a reduced amount of gene product is made in the cell such that the total functional activity of the gene product in the cell is reduced as compared to expression of a wildtype version of the gene, or an altered gene product with altered (e.g., reduced) functional activity is made in the cell as compared to expression of a wildtype version of the gene.

As used herein, the term “stop codon mutation” is intended to refer to a mutation that results in the introduction of a non-naturally-occurring stop codon in the transcribed region of the gene.

As used herein, the term “missense mutation” is intended to refer to a mutation that results in the introduction of an amino-acid substitution at the mutated codon position, or the introduction of a non-naturally-occurring stop codon at the mutated codon position (i.e., as used herein the term “missense mutation” includes stop codon mutations as well as mutations leading to amino acid substitutions).

As used herein, the term “contacting the eukaryotic cell with an agent” refers to exposure of the eukaryotic cell to an agent, either directly or indirectly, such as by addition of the agent to culture medium in which the cell is growing, administration of the agent to an organism comprising the cell, introduction of the agent into the cell or stimulating expression of the agent within the cell. The term is further intended to mean that the agent is able to be made available, or is active, at the interior of the cell where gene translation is occurring.

As used herein, the term “agent” refers to a molecule, including antibiotics, proteins, polypeptides, and the like, which can be administered as needed to the interior of a eukaryotic cell used in the method of the invention, and whose presence is able to suppress mutations of the invention.

As used herein, the term “suppress a mutation” is intended to encompass counteracting, or overcoming, the effect of the mutation, either partially or entirely, such that translation of the gene is partially or fully restored and/or expression of a functional gene product encoded by the gene is partially or fully restored.

The invention provides a method of regulating expression of a gene in a eukaryotic cell which method comprises:

introducing at least one mutation into a transcribed region of the gene such that translation of the gene terminates at or is altered at the at least one mutation and a functional gene product encoded by the gene is not produced in the cell; and

contacting the eukaryotic cell with an agent that suppresses the at least one mutation of the gene such that a functional gene product of the gene is produced in the eukaryotic cell, thereby regulating expression of the gene in the eukaryotic cell.

In one embodiment, the at least one mutation that is introduced into the gene creates at least one stop codon in a coding region of the eukaryotic gene. In this embodiment, the mutation(s) can be suppressed using an agent that is an aminoglycoside. In another embodiment, the at least one mutation introduced into the gene creates at least one missense mutation in a coding region of the eukaryotic gene. In this embodiment, the mutation(s) can be suppressed using an agent that is a suppressor tRNA. In yet another embodiment, the at least one mutation introduced into the gene creates a ribozyme cleavage site that is cleaved by a ribozyme in the eukaryotic cell. In this embodiment, the mutation(s) can be suppressed using an agent that is an inhibitor of the ribozyme that cleaves the ribozyme cleavage site.

In preferred embodiments, the gene to be regulated is a eukaryotic gene, a viral gene or a gene of therapeutic interest.

In the following subsections, various embodiments of the regulatory methods of the invention, as well as the nucleic acids, polypeptides and chemical agents comprising the components of the inducible expression system of the invention, and their interrelationship, are discussed in greater detail.

I. Modification Of A Eukaryotic Gene To Introduce Appropriate Mutations

A. Types of Genetic Modifications for use in the Invention

The triplet genetic code is used by eukaryotic organisms to determine the sequence of amino acids incorporated during protein synthesis. The use of a specific set of codons to specify the incorporation of each amino acid is highly conserved among most organisms. In keeping with this conservation, translation is most always initiated at a methionine codon (AUG) codon, and the end of translation is signaled by one of three possible stop codons (UAG, UAA, UGA). In the inducible expression system of the invention, the expression of a desired gene in a eukaryotic cell is perturbed by purposely introducing at least one nucleotide base pair mutation into the coding sequence of that gene to thereby perturb normal translation of the gene. Such mutations may introduce a stop or missense mutation into the messenger RNA. These mutations typically manifest themselves as truncated or altered versions of the encoded gene product, and mutations are chosen such that an inactive (partially or completely) gene product is produced in the cell. Still other mutations result in alterations of the kinetics of messenger RNA stability, which reduce or eliminate the amount of available message for translation, and, thus, the amount of functional gene product produced in the cell. Thus, the deliberate mutations used in the inducible expression system of the invention prevent production of the functional product of the desired gene in a eukaryotic cell under normal conditions.

One kind of genetic mutation that can be used in the invention is a stop codon mutation, whereby one of the three stop codons (i.e., UAG, UGA, UAA) is created from a single, double, or triple nucleotide mutation in the nucleotides of one or more selected amino acid-specifying codons. Genes that have such mutations introduced into their coding region will, upon translation, produce gene products that are terminated at the codon of the mutation. Many of such truncated gene products are inactive, e.g. if the stop codon mutation occurs near the 5′ end of the coding region, and, accordingly, one or more stop codon mutations that lead to an inactive (partially or completely) gene product can be chosen for use in the method of the invention. Thus, in one embodiment of the inducible expression system of the invention, one or more stop codon mutations are introduced into the coding region of a desired gene, i.e, a codon for an amino acid in the encoded gene product is deliberately converted to a stop codon.

The Examples section below describes in detail the construction of various mutants of the human erythropoietin gene in which stop codon mutations have been introduced at various different positions of the coding region, and assessment of the expression, and inducibility, of such mutant constructs. Preferred mutated Epo constructs of the invention include constructs in which the human Epo cDNA is mutated at nucleotide position 199, 208, 214, 307, 434, 437, 454 or 526, or in which the human Epo cDNA is mutated at a codon encoding amino acid position 6, 9, 11, 62, 85, 86, 91 or 115. More preferred constructs are those mutated at nucleotide position 199, 208 or 214, or at a codon encoding amino acid position 6, 9 or 11. Similar methodology to that described in the Examples can be used to assess stop codons introduced into other genes, or into other locations of the Epo gene, to allow for selection of appropriate stop codon mutations that lead to an inactive (partially or completely) gene product, yet which can be suppressed by an agent of the invention (such as an aminoglycoside).

Another kind of genetic mutation that can be used in the invention is a missense mutation. As used herein, a missense mutation refers to a single, double or triple nucleotide change in the nucleotides of an amino acid-specifying codon in the coding region of a gene that results in introduction of a different amino acid residue, or no amino acid residue, at that position, relative to the wildtype version of the gene (e.g., the missense mutation may result in an amino acid substitution or premature termination of translation). The genetic code is used to determine which nucleotide change or changes are to be made to result in a particular amino acid mutation or mutations. Genes that have such missense mutations introduced into their coding region will produce gene products that have an alternative (i.e. non-wild type) amino acid at that codon position in the nascent polypeptide chain such that the mutant polypeptides may have activity that is altered or diminished or more susceptible to cellular proteases. Alternatively, such mutated gene products may be completely inactive. Accordingly, one or more missense mutations that lead to an inactive (partially or completely) gene product or functionally altered gene product can be chosen for use in the method of the invention. Thus, in another embodiment of the inducible expression system of the invention, a missense mutation is deliberately introduced into the coding region of a desired gene, i.e., a codon for an altered amino acid, or a stop codon, is deliberately introduced at one or more predetermined positions.

A third kind of genetic mutation that can be used in the invention is a mutation of the coding region of a gene that results in the introduction or improvement of a ribozyme cleavage site that is cleaved by a ribozyme. The introduction of such a site can significantly reduce the stability of an RNA message, e.g. by creating a ribozyme recognition site or by increasing the activity of a ribozyme recognition site that is already present. Such mutations can result in expression of the desired gene product being diminished or arrested. Thus, in another embodiment of the inducible expression system of the invention, a ribozyme cleavage site is introduced into the transcribed region of the desired gene. The nature of the mutation is determined by the specificity of the ribozyme that will be used to cleave the RNA message. Site-specific cleavage of a particular RNA sequence by a particular ribozyme has been extensively studied in the art, and numerous ribozymes have been designed that target specifically a particular mRNA (see e.g., Macejak, D.G., et al. (1999) J. Virol. 73:7745-7751; Couture, L.A. and Stinchcomb, D.T. (1996) Trends Genet. 12:510-515; Bramlage, B. et al. (1998) Trends Biotechnol. 16:434-438; Muotri, A.R. et al. (1999) Gene 237:303-310; and Welch, P.J. et al. (1998) Curr. Opin. Biotechnol. 9:486-496). Previously, however, these ribozymes typically have been designed to cleave a naturally-occurring mRNA that encodes a protein whose activity is to be ablated for therapeutic purposes (referred to as “anti-gene therapy”), for example ribozymes that cleave naturally-occurring oncogene or viral gene sequences. In contrast, in the present invention this same type of ribozyme technology is applied to cleavage of mutated mRNA sequences, wherein the mutation has been intentionally introduced into the desired gene for regulatory purposes. Thus, a ribozyme is designed, using information and technology well-known in the art, that recognizes a mutated form of the desired gene, and then this corresponding mutation comprising the ribozyme cleavage site is introduced into the desired gene (e.g., by methodologies described in the following subsection) such that mRNA transcribed from the desired (now mutated) gene is now capable of being recognized, and cleaved, by the ribozyme. The ribozyme responsible for cleaving mRNA of the mutated desired gene may be constitutively present in cells expressing the desired gene or, alternatively, may be introduced into the cells. A preferred method for introducing and expressing the ribozyme into the cells of interest is by adenoviral-mediated expression (see e.g.,. Macejak, D.G. et al. (1999) J. Virol. 73:7745-7751). Alternatively, other methods available in the art for introducing and expressing nucleic acid in host cells (such as those described in subsections ID and IE below) can be used.

B. Methods of Introducing a Mutation into a Gene of Interest

The inducible expression system of the invention involves, at least in part, the deliberate introduction of nucleotide mutations into the coding region of a desired gene. The nature of such mutations of the invention has been outlined in the preceding section. A genetic mutation of the invention may be introduced into the coding region of a desired gene by any one of a number of different ways known in the art. Some or all of the nucleotide sequence of the coding region of the desired gene must be known to allow for design of the appropriate introduced mutation(s). For desired genes in which the sequence of some or all of the coding region is not known, standard DNA sequencing methods can be used to obtain the sequence of some or all of the coding region of the gene.

In one embodiment of the invention, mutations of the invention are introduced into a desired gene through methods of directed mutagenesis. As used herein, the term “directed mutagenesis” (also referred to as site-directed mutagenesis) encompasses methods of introducing a specific and predetermined mutation into a known nucleotide sequence. Furthermore, as used herein, the term “directed mutagenesis” implies that some or all of the nucleotide sequence that is intended to be mutated has been determined or is known. For example, one or more specific mutations may be introduced into the coding region of a desired gene by one skilled in the art using directed mutagenesis methods involving specifically designed oligonucleotides, e.g. oligonucleotide-directed mutagenesis. These methods typically involve the introduction of the desired gene into a vector that can be reliably replicated from an oligonucleotide primer. Oligonucleotide primers with one or more nucleotide mismatches inside the hybridizing region (i.e., the region that hybridizes to the desired gene) can be readily designed and used by one skilled in the art to introduce mutations of the invention into a desired gene by standard recombinant DNA techniques known in the art

In one embodiment of the invention, a viral vector replication system is used for oligonucleotide-directed mutagenesis, e.g. an M13 viral vector. Methods for introducing mutations of the invention with M13 and other viral vectors are readily available in the art (see, for example, Kunkel, 1985, PNAS U.S.A., 82:488-492; Kunkel et al, 1987, Meth. Enzymol., 154:367-382).

In another embodiment of the invention, a bacterial plasmid replication system is used, in which the desired gene is introduced into the bacterial plasmid and the mutation(s) of the invention is deliberately introduced into the desired gene with oligonucleotide primers containing one or more mismatches at preselected nucleotide positions. Several such systems have been described that can be readily used by one skilled in the art (see, for example, Deng and Nikoloff, 1992, Anal. Biochem. 200:81-88; Nikoloff et al, 1996, 58:455-468).

In a preferred embodiment of the invention, mutations of the invention may be introduced into desired genes during amplification with PCR (polymerase chain reactions), also referred to as PCR-mediated mutagenesis. A number of PCR-based methods to deliberately introduce mutations into a desired gene are available in the art and can be used in the invention (see, for example, Ausubel et al, Current Protocols in Molecular Biology, John Wiley & Sons, 1995). These methods generally involve the use of oligonucleotide primers for PCR amplification reactions that contain one or more nucleotide mismatches within the hybridizing region (i.e., the region of hybridization with the desired gene). Example 1, below, describes further the use of PCR-mediated mutagenesis to introduce various stop codon mutations into the human erythropoietin gene.

Another approach to the generation of mutations of the invention in a desired gene is through methods of non-directed mutagenesis. As used herein, the term “non-directed mutagenesis” refers to any method of introducing mutations randomly into the desired gene in a replicable biological system and having a means of screening resultant nucleic acids and to select and use the one(s) which contains a mutation of the invention in a desired gene. Such non-directed mutagenesis can be accomplished through a variety of means, for example, by exposure of the gene and/or the replicable biological system in which it is propagated, to mutagenic conditions, e.g. irradiation, chemical mutagens and the like. Other methods available to one skilled in the art include any method involving enzymatic replication of nucleic acid, for example with a polymerase or a replicase, whereby the intrinsic replication error rate of the enzyme is able to accomplish non-directed mutagenesis. As used herein, “screening” of the resultant nucleic acids to find a desired gene with a mutation of the invention includes methods for determining nucleotide sequences, e.g. sequencing analysis. In addition, the term “screening” also includes methods for determining amino acid sequences of protein, e.g. microsequencing. The term “screening” furthermore includes biological assays that can discern mutations of the invention in a desired gene, in particular mutations that result in the production of an inactive (partially or completely) gene product upon translation of the desired gene. Depending on which desired gene is selected, the ordinarily skilled artisan can select an appropriate known biological assay by which to screen randomly generated mutants to thereby select mutants that result in the production of an inactive (partially or completely) gene product upon translation of the desired gene.

In still another embodiment, a mutation of the invention is introduced into a desired gene that is present within the genome of a eukaryotic cell (i.e., an endogenous gene) by manipulation of the endogenous gene sequence, preferably by homologous recombination. Methods of using homologous recombination to alter the sequence of an endogenous eukaryotic gene are well known in the art and can be applied to deliberately introduce one or more mutations of the invention into a desired endogenous eukaryotic gene. Such methods typically involve construction of a gene targeting vector comprising at least a portion of the endogenous gene sequence, introduction of the targeting vector into a eukaryotic cell containing the endogenous desired gene and culture under conditions sufficient to allow for homologous recombination between the targeting vector and the endogenous gene, followed by screening of clones for ones in which the endogenous gene has undergone homologous recombination with the targeting vector using standard recombinant DNA techniques. To introduce one or more mutations into the endogenous gene, the targeting vector includes at least a portion of the endogenous gene sequence into which the mutation(s) has been introduced, flanked by wildtype sequences of the endogenous gene to facilitate homologous recombination between the targeting vector and the endogenous gene.

C. Desired Genes of the Invention

The inducible expression system of the invention can be applied to essentially any gene whose coding region sequence (all or a part) is known or can be determined (e.g, by sequencing), and can be applied to isolated genes (e.g., nucleic acid fragments, or vectors containing the gene, in vitro) as well as to endogenous genes in eukaryotic cells. However, due to the ease in the ability to manipulate them (e.g., mutate them in vitro), isolated genes are somewhat preferred for use in the invention. As used herein, the terms “desired gene” and “gene of the invention” refer to a gene that is expressed in the inducible expression system of the invention, or in any of the embodiments of the invention described herein. As used herein, the term “isolated gene” refers to a nucleic acid molecule that corresponds to part or all of a gene that has been removed from its genomic context. An isolated gene may be propagated by techniques that are generally known in the art, e.g. as part of a replicable biological vector, e.g. a viral vector, a bacterial plasmid, or a cosmid. Isolated genes are able to be deliberately mutated using standard techniques of molecular biology that are generally known in the art.

There are many methods in the art that can be used to obtain an isolated gene for use as a gene of the invention. In a preferred embodiment, the desired gene is obtained in the form of a cDNA, although genomic DNA also can be used. An isolated cDNA for a known gene (i.e., a gene whose nucleotide sequence is available in the art) can be obtained by, for example, amplifying the cDNA by PCR using mRNA from a source known to express the gene (e.g., a cell line or tissue known to express the gene), such as by standard reverse-transcriptase PCR. In another embodiment, a desired gene is obtained as cDNA from an appropriate expression library, i.e. an expression library that is expected to include the desired gene. Numerous standard cloning methods are available in the art for obtaining a desired gene when one or more of the physical properties of the cDNA corresponding to the desired gene are known, e.g. full or partial sequence information, approximate number of nucleotide base pairs, or a map of cleavage sites of restriction enzymes.

In another embodiment, the desired gene is an isolated gene that contains some or all of its exons and introns. Numerous methods are available in the art for the isolation of such a gene from its genomic context such as cloning from a genomic DNA library, and the further manipulation of some or all of the gene as it relates to the invention, e.g. mutagenesis (as described in the subsection above).

In a preferred embodiment, the desired gene is a eukaryotic gene, more preferably a human gene, and even more preferably a gene of therapeutic interest, such as a human gene of therapeutic interest.

In another embodiment, the desired gene is obtained from a non-eukaryotic organism, e.g. a prokaryote or a virus (e.g., a herpes simplex virus or a retrovirus), with the proviso that the gene is not the bacterial chloramphenicol acetyltransferase gene. Numerous methods are available in the art for the isolation of such a gene from the genome of such organisms including PCR amplification, and for the further manipulation of the gene as it relates to the invention, e.g. mutagenesis (as described in the subsection above).

In another embodiment, the desired gene is created in whole or in part with nucleic acids that are synthesized, e.g. with an oligonucleotide synthesis apparatus or protocol. There are numerous methods available to one skilled in the art that allow for the generation of a desired gene, at least part of which is deliberately synthesized. In the hands of one skilled in the art, such synthesized nucleic acids can be used wholly as the desired gene of the invention, or can be combined with nucleic acids from other sources (e.g. expression libraries, etc). Such combinations of nucleic acids from synthetic sources and from biological sources can be readily achieved with a variety of methods known to one skilled in the art, e.g. ligation.

In a preferred embodiment, the desired gene is a gene of therapeutic interest. As used herein, a gene of “therapeutic interest” is intended to encompass genes whose expression can ameliorate a disease condition or disorder. A gene of therapeutic interest may act by supplying a gene product of therapeutic benefit (e.g., replacement of a defective gene product in a cell or supplementing a defective gene product in a cell) or by inhibiting an undesireable process or activity, such as inhibiting the growth or spread of cancer cells.

In a preferred embodiment, the desired gene is an erythropoietin gene. In a further embodiment, the methods of the present invention are used to treat a subject who has a disorder characterized by aberrant (e.g., deficient) erythropoietin polypeptide or nucleic acid expression. In a preferred embodiment, the disorder is an erythropoietin responsive anemia. The utility of erythropoietin for treatment of such disorders, using methodology distinct from that of the invention described herein, has been discussed elsewhere (for example, see Goodnough et al, 1997, New England J. of Med., 336:933-938). One skilled in the art has ample access to resources and methods for utilization of an erythropoietin gene as the gene of the invention, as outlined above. For example, the nucleotide sequence for human erythropoietin can be found under Accession Number NM000799 of the public GenBank database (Jacobs et al, 1985, Nature, 313:806-810), and PCR primers can be designed based on this publicly-available sequence that allow for amplification, and thereby isolation, of human erythropoietin cDNA Moreover, appropriate mutations to be introduced into the Epo gene (e.g., the human Epo gene) can be designed based on the known sequence of the Epo gene.

In another embodiment, the desired gene is an insulin gene. In a further embodiment, the methods of the present invention are used to treat a subject who has a disorder characterized by aberrant (e.g., deficient) insulin polypeptide or nucleic acid expression. In a preferred embodiment, the disorder is type 1 diabetes or diabetes mellitus. The utility of insulin for treatment of such disorders, using methodology distinct from that of the invention described herein, has been discussed elsewhere (for example, see Lorenz, 1999, Prim. Care, 26:917-929). One skilled in the has ample access to resources and methods for utilization of an insulin gene as the gene of the invention, as outlined above. For example, the nucleotide sequence for human insulin can be found under Accession Number M10039 of the public GenBank database, and PCR primers can be designed based on this publicly-available sequence that allow for amplification, and thereby isolation, of human insulin cDNA. Moreover, appropriate mutations to be introduced into the insulin gene (e.g., the human insulin gene) can be designed based on the known sequence of the insulin gene.

In another embodiment, the desired gene is a VEGF (vascular endothelial growth factor) gene. As used herein, the term “VEGF” is intended to include the family of VEGF proteins, not merely one particular VEGF protein. In a further embodiment, the methods of the present invention are used to treat a subject who has a disorder characterized by aberrant VEGF polypeptide or nucleic acid expression or in which upregulation of VEGF expression is desired (e.g., wherein therapeutic angiogenesis is desired). In a preferred embodiment, the disorder is a cardiovascular disorder (e.g., a subject in need of cardiac neovascularization), or a related disorder in which neovascularization is beneficial. The utility of VEGF for treatment of such disorders using methodology distinct from that of the invention described herein, has been discussed elsewhere (for example, see Losordo et al 1999, Am Heart J., 138:132-141; Battegay 1995, J. Mol. Med., 73:333-346). One skilled in the art has ample access to resources and methods for utilization of an VEGF gene as the gene of the invention, as outlined above. For example, the nucleotide sequence for human VEGF can be found under Accession Number E15157 of the public GenBank database, and PCR primers can be designed based on this publicly-available sequence that allow for amplification, and thereby isolation, of human VEGF cDNA. Moreover, appropriate mutations to be introduced into the VEGF gene (e.g., the human VEGF gene) can be designed based on the known sequence of the VEGF gene.

In another embodiment, the desired gene is an FGF (fibroblast growth factor) gene. As used herein, the term “FGF” is intended to include the family of FGF proteins, not merely one particular FGF protein. In a further embodiment, the methods of the present invention are used to treat a subject who has a disorder characterized by aberrant FGF polypeptide or nucleic acid expression or in which upregulation of an FGF polypeptide expression is desired (e.g., wherein therapeutic angiogenesis is desired). In a preferred embodiment, the disorder is a cardiovascular disorder (e.g., a subject in need of cardiac neovascularization), or a related disorder in which neovascularization is beneficial. The utility of FGF for treatment of such disorders, using methodology distinct from that of the invention described herein, has been discussed elsewhere (for example, see Lewis et al, 1997, Cardiovasc. Res., 35:490-497; Battegay 1995, J. Mol. Med., 73:333-346). One skilled in the art has ample access to resources and methods for utilization of an FGF gene as the gene of the invention, as outlined above. For example, the nucleotide sequence for human FGF can be found under Accession Number NM_{—}004464 of the public GenBank database, and PCR primers can be designed based on this publicly-available sequence that allow for amplification, and thereby isolation, of human FGF cDNA. Moreover, appropriate mutations to be introduced into the FGF gene (e.g., the human FGF gene) can be designed based on the known sequence of the FGF gene.

In another embodiment, the desired gene is an HIF-1α (hypoxia-inducible factor 1 alpha) gene. In a further embodiment, the methods of the present invention are used to treat a subject who has a disorder characterized by aberrant HIF-1α polypeptide or nucleic acid expression or in which upregulation of HIF-1α polypeptide expression is desired (e.g., wherein therapeutic angiogenesis is desired). In a preferred embodiment, the disorder is a cardiovascular disorder (e.g., a subject in need of cardiac neovascularization), or a related disorder in which neovascularization is beneficial. The utility of HIF-1α for treatment of such disorders, using methodology distinct from that of the invention described herein, has been discussed elsewhere (for example, see Lewis et al, 1997, Cardiovasc. Res., 35:490-497; Ravi et al, 2000, Genes Dev. 14:34-44; Harris et al, 1996, Breast Cancer Res. Treat., 38:97-108). One skilled in the art has ample access to resources and methods for utilization of an HIF-1α gene as the gene of the invention, as outlined above. For example, the nucleotide sequence for human HIF-1α can be found under Accession Number AH006957 of the public GenBank database, and PCR primers can be designed based on this publicly-available sequence that allow for amplification, and thereby isolation, of human HIF-1α cDNA. Moreover, appropriate mutations to be introduced into the HIF-1α gene (e.g., the human HIF-1α gene) can be designed based on the known sequence of the HIF-1α gene.

Other therapeutic genes of interest that can be used in the invention include those encoding anti-angiogenesis factors, such endostatin or angiostatin. The nucleotide sequence for human endostatin can be found under Accession Number AF184060 of the public GenBank database, whereas the nucleotide sequence for human angiostatin can be found in U.S. Pat. No. 5,837,682. PCR primers can be designed based on these publicly-available sequences that allow for amplification, and thereby isolation, of human endostatin or angiostatin cDNA. Moreover, appropriate mutations to be introduced into the human endostatin or angiostatin gene can be designed based on the known sequences of the human endostatin and angiostatin genes.

In another embodiment, the desired gene is a coagulation factor gene. In a further embodiment, the desired gene is a Factor VIII (coagulation factor 8) gene. In yet another embodiment, the methods of the present invention are used to treat a subject who has a disorder characterized by aberrant (e.g., deficient) Factor VIII polypeptide or nucleic acid expression. In another embodiment, the desired gene is a Factor IX (coagulation factor 9) gene. In yet another embodiment, the methods of the present invention are used to treat a subject who has a disorder characterized by aberrant (e.g., deficient) Factor IX polypeptide or nucleic acid expression. In a preferred embodiment, the disorder is hemophilia, a blood coagulation disorder, or a related disorder. The utility of coagulation factors (Factor VIII and Factor IX) for treatment of such disorders, using methodology distinct from that of the invention described herein, has been discussed elsewhere (for example, see Kaufman, 1999, Hum. Gen. Ther., 10:2091-2107). One skilled in the art has ample access to resources and methods for utilization of a Factor VIII gene or a Factor IX gene as the gene of the invention, as outlined above. For example, the nucleotide sequence for human Factor VIII can be found under Accession Number NM_{—}000132 of the public GenBank database and the nucleotide sequence for human Factor IX can be found under Accession Number NM_{—}000133 of the public GenBank database, and PCR primers can be designed based on this publicly-available sequence that allow for amplification, and thereby isolation, of human Factor VIII or IX cDNA. Moreover, appropriate mutations to be introduced into the Factor VIII or IX gene (e.g., the human Factor VIII or IX gene) can be designed based on the known sequence of the Factor VIII or IX gene.

In another embodiment, the desired gene is a GH (growth hormone) gene (e.g., a human growth hormone gene). In a further embodiment, the methods of the present invention are used to treat a subject who has a disorder characterized by aberrant (e.g., deficient) GH polypeptide or nucleic acid expression. In a preferred embodiment, the disorder is pituitary dwarfism, wasting disorders, or related disorders. The utility of GH for treatment of such disorders, using methodology distinct from that of the invention described herein, has been established in the art. One skilled in the art has ample access to resources and methods for utilization of a GH gene as the gene of the invention, as outlined above. For example, the nucleotide sequence for human GH can be found under Accession Number NM_{—}000515 of the public GenBank database, and PCR primers can be designed based on this publicly-available sequence that allow for amplification, and thereby isolation, of human GH cDNA. Moreover, appropriate mutations to be introduced into the GH gene (e.g., the human GH gene) can be designed based on the known sequence of the GH gene.

In another embodiment, the desired gene is a HSVTK (Herpes Simplex Virus Thymidine Kinase) gene, which can be used as a “suicide gene” to kill cells into which the gene is introduced (e.g., malignant cells) upon treatment with an appropriate drug (e.g., gancyclovir). In a preferred embodiment, the disorder is a cancer or malignant cell condition or restenosis or other vascular proliferative disorders, or a related disorder. The utility of HSVTK as a “suicide gene” to kill cells into which it has been introduced has been established in the art. One skilled in the art has ample access to resources and methods for utilization of an HSVTK gene as the gene of the invention, as outlined above. For example, the nucleotide sequence for human HSVTK can be found under Accession Number AB009254 of the public GenBank database, and PCR primers can be designed based on this publicly-available sequence that allow for amplification, and thereby isolation, of human HSVTK cDNA. Moreover, appropriate mutations to be introduced into the HSVTK gene (e.g., the human HSVTK gene) can be designed based on the known sequence of the HSVTK gene.

D. Transcriptional Expression of the Desired Gene

Various embodiments of the inducible expression system described herein require the use of vectors, such as expression vectors. As used herein, the terms “vector” and “expression vector” refer to a nucleic acid that is capable of replication within at least one biological system. An expression vector may be for example, a bacterial nucleic acid (e.g. a plasmid), a viral nucleic acid, or a eukaryotic nucleic acid. An expression vector may be a combination of nucleic acids from bacterial, eukaryotic and/or viral systems. As used herein, a vector is combined (e.g. ligated, spliced) with some or all of the gene of interest. One embodiment of the invention that involves at least one vector is outlined above, wherein the desired gene is incorporated (e.g. ligated, spliced) into a vector for the purpose of introducing one or more mutations of the invention.

As further used herein, the term “expression vector” refers to nucleic acids of eukaryotic, bacterial, or viral origin, into which a desired gene can be incorporated, and that are thereafter capable of introduction into a host eukaryotic cell. Expression vectors in this embodiment may also refer to combinations of nucleic acids of eukaryotic, bacterial, and/or viral origin. Expression vectors may also, but need not necessarily, become integrated into the genome of the host. As used herein, the term “expression vector” also refers to nucleic acids that can support the expression in the host cell of a desired gene that has been incorporated into the expression vector.

In further embodiments, the expression vectors of the system may contain one or more of the following genetic elements to support expression of the desired gene, one or more enhancers, one or more promoters, and one or more polyadenylation sites.

As used herein, the term “enhancer” refers to a nucleic acid sequence that is able to increase the transcription of a gene located elsewhere on the same nucleic acid molecule, or on a different nucleic acid molecule. Enhancers typically are of eukaryotic or viral origin. Enhancers often contain palindrome or near-palindrome nucleotide sequence. Enhancers can be effective when placed very near the start site of a gene (e.g. a gene of the invention) or very far upstream or downstream of the transcriptional start site. Some enhancers will function from within the coding sequence of a gene. Although enhancers may contain asymmetric sequences (i.e. non-palindrome sequence), their alignment in relation to the affected gene often appears to be unimportant for their transcriptional function. Enhancers may be functional in many cell types or only in certain cell types (e.g. tissue-specific enhancers). In a preferred embodiment, an enhancer used in the invention is the human immunoglobulin gene 4F2 enhancer (Karpinski et al, 1989, Mol. Cell Biol., 9:2588-2597 ). In another preferred embodiment, an enhancer used in the invention is the CMV (cytomegalovirus) enhancer. In another preferred embodiment, an enhancer used in the invention is a RSV (rous sarcoma virus) enhancer. In another preferred embodiment, an adenoviral enhancer is used.

As used herein, the term “promoter” refers to a nucleic acid sequence that is necessary for transcription of a nearby gene. Promoters can be, for example, of eukaryotic, viral or of bacterial origin. Promoters are of diverse structure and can contain many or few discernible genetic elements. Enhancers may be included within promoters. In order to function, promoters normally need to be located directly adjacent to or near the affected gene. Promoters may be functional in many cell types or only in certain cell types (e.g. tissue-specific promoters). In a preferred embodiment, a promoter of the invention is the human EF-1α (elongation-1 alpha) promoter. In another preferred embodiment, a promoter of the invention is the CMV promoter. In another preferred embodiment, an RSV promoter is used. In another preferred embodiment, an adenoviral promoter is used.

As used herein, the term “polyadenylation site” refers to a discrete nucleotide sequence at the 3′ end of a transcribed gene which causes the RNA transcriptional machinery to cleave the nascent mRNA molecule and to add multiple adenylribonucleotides to the 3′ end of the mRNA molecule. This feature is part of eukaryotic mRNA processing and is often included in expression vectors to increase the stability of an mRNA from a desired gene. Often the polyadenylation signal includes the consensus sequence AAUAAA. In a preferred embodiment, the polyadenylation site of the bovine growth hormone (BGH) gene is used. In another preferred embodiment, an adenoviral polyadenylation site is used.

When used in mammalian cells, a recombinant expression vector's control functions are often provided by viral genetic material. For example, commonly used promoters are derived from polyoma, Adenovirus 2, cytomegalovirus, rous sarcoma virus and Simian Virus 40. Use of viral regulatory elements to direct transcriptional expression of the desired gene can allow for high level constitutive transcription of the gene in a variety of host cells. In one embodiment of a recombinant expression vector, the sequences encoding the desired gene are flanked upstream (i.e., 5′) by the human cytomegalovirus IE promoter and downstream (i.e., 3′) by an SV40 poly(A) signal. The human cytomegalovirus IE promoter is described in Boshart et al. (1985) Cell 41:521-530. Other ubiquitously expressing promoters which can be used include the HSV-Tk promoter (disclosed in McKnight et al. (1984) Cell 37:253-262), RSV promoter, and β-actin promoters (e.g., the human β-actin promoter as described by Ng et al. (1985) Mol. Cell. Biol. 5:2720-2732).

Alternatively, the regulatory sequences of the recombinant expression vector can direct transcriptional expression of the desired preferentially in a particular cell type, i.e., tissue-specific regulatory elements can be used. Non-limiting examples of tissue-specific promoters which can be used include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

E. Transfer of Genes of the Invention into Host Cells

The inducible expression system of the invention is based, at least in part, upon the expression of a desired gene in a host cell. In certain embodiments of the invention, the desired gene must be transferred into (i.e., introduced into) the host cell and, in some cases, into the host cell nucleus. As used herein, the term “introduced into” refers to a methodology of putting one or more nucleic acid molecules of the invention (e.g. expression vector with an incorporated desired gene) into the host cells. As mentioned above, a host cell may be a cultured eukaryotic cell, or a cell that is part of a tissue of an organism, or may itself be an organism (e.g. a yeast cell).

In one embodiment of the invention, transfer is brought about by use of one of a variety of different transfection methods well known in the art. As used herein, “transfection” refers to a methodology of deliberately delivering nucleic acid molecules of the invention into cultured host cells Suitable transfection methods include calcium phosphate precipitation (in which calcium phosphate aggregates that include a gene of the invention are incubated with the host cells), lipofection (in which a gene of the invention is combined with a liposome reagent and applied to host cells) and electroporation (in which a gene of the invention, in aqueous solution, is taken up by the host cells when a pulsed electrical field is applied).

In another embodiment of the invention, transfer is brought about by allowing the host cells to become infected with a viral vector that contains the gene of interest. In such an embodiment, the expression vector that incorporates the desired gene is, at least partly, a viral vector. Types of viral vectors that can be used to deliver the gene into the host cells by viral infection include retroviral vectors, lentiviral vectors, adenoviral vectors and adeno-associated viral vectors. Viral infection can be used, for example, to deliver a gene of the invention into cultured host cells in vitro or into cells in tissues of an organism in vivo.

In another embodiment, the nucleic acid molecules of the invention are transferred directly into cultured host cells using a mechanical delivery system, such as by way of a “gene gun”, including bombardment of the cell with vector-coated microparticles. Moreover, in another embodiment, the nucleic acid molecules of the invention are transferred directly into host tissue cells in vivo (e.g., into muscle cells of a host organism in vivo) using a mechanical delivery system, such as by way of a “gene gun”. As used herein, a “gene gun” system refers to technology that can directly transfer genetic material into cultured cells or into tissue cells by a minute explosive charge that projects DNA- or RNA-coated microparticles into the cells.

The steps of the invention for introducing a mutation(s) into a desired gene, optionally introducing the desired gene into a cell (e.g., for isolated genes) and suppressing the mutation(s) in the desired gene using the agent, in a cell or a subject, can be varied out in various ways. For example, in one embodiment, a mutation is introduced into an isolated gene in vitro, the now-mutated gene is introduced into a cell in culture and then the agent is added to the culture to suppress the mutation in the gene, thereby modulating expression of the gene in cells in culture. In another embodiment, a mutation is introduced into an isolated gene in vitro, the now-mutated gene is introduced into a cell in culture, the cultured cells are administered to a subject and then the agent is administered to the subject to suppress the mutation in the gene, thereby modulating expression of the gene in the subject. In yet another embodiment, a mutation is introduced into an isolated gene in vitro, the now-mutated gene is introduced into a cells in a subject (e.g., by direct delivery or viral delivery) and then the agent is administered to the subject to suppress the mutation in the gene, thereby modulating expression of the gene in the subject.

For regulation of endogenous genes, in one embodiment, a mutation is introduced into an endogenous gene of a cell in culture and then the agent is added to the culture to suppress the mutation in the gene, thereby modulating expression of the gene in cells in culture. In another embodiment, a mutation is introduced into an endogenous gene of a cell in culture, the cultured cells are administered to a subject and then the agent is administered to the subject to suppress the mutation in the gene, thereby modulating expression of the gene in the subject. In yet another embodiment, a mutation is introduced into an endogenous gene of a subject and then the agent is administered to the subject to suppress the mutation in the gene, thereby modulating expression of the gene in the subject.

For administration of cells to a subject, it may be preferable to first remove residual compounds in the culture from the cells before administering them to the subject. This can be done for example by gradient centrifugation of the cells or by washing of the tissue. For further discussion of ex vivo genetic modification of cells followed by readministration to a subject, see also U.S. Pat. No. 5,399,346 by W. F. Anderson et al.

For in vivo regulation of gene expression in a subject, a desired gene can be introduced into cells of a subject using methods known in the art for introducing nucleic acid (e.g., DNA) into cells in vivo. Examples of such methods include:

Direct Injection: Naked DNA can be introduced into cells in vivo by directly injecting the DNA into tissues (see e.g., Acsadi et al. (1991) Nature 332:815-818; Wolff et al. (1990) Science 247:1465-1468). In a distinct method, a delivery apparatus (e.g., a “gene gun”) for injecting DNA into cells in vivo can be used. Such an apparatus is commercially available (e.g., from BioRad).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C.H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A.D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816), muscle cells (Quantin et al. (1992) Proc. Natl. Acad. Sci. USA 89:2581-2584), and cardiomyocytes (Barr et al. (1994) Gene Therapy 1:51-58). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular transcriptional expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product (e.g., before and after gene induction using the agent that suppresses the introduced mutation(s)) can be detected by an appropriate assay, for example by immunological detection of a produced protein, such as with a specific antibody, or by a functional assay to detect a functional activity of the gene product, such as an enzymatic assay.

II. Regulation of Gene Expression Using the System of the Invention

In the regulatory methods of the invention, after one or more mutations have been introduced into a transcribed region of a gene of interest, the expression of that gene of interest in a eukaryotic cell is modulated by contacting the cell with an agent that suppresses the mutation(s), such that a functional gene product is produced in the cell.

A. Aminoglycosides

In one embodiment, wherein a stop codon mutation(s) has been introduced into the gene, the agent is an aminoglycoside. Aminoglycosides are a class of compounds with antibiotic properties. It is known that at least some of their antibiotic effect occurs through a mechanism whereby translation at the ribosomal subunits is disrupted. Aminoglycosides preferentially bind to the 30S eukaryotic ribosomal subunit. At certain subtoxic concentrations, the partial effect of aminoglycosides on the translation machinery is to reduce the fidelity of the translation machinery, such that codons for termination are misread to be codons for amino acids (e.g. tyrosine). It is this property of the aminoglycosides that make them useful in the preferred embodiments of the inducible expression system described herein. In a preferred embodiment, the aminoglycoside agent is Hygromycin-B. In another preferred embodiment, the aminoglycoside agent is Paromomycin. In another preferred embodiment, the aminoglycoside agent is Tobramycin. In another preferred embodiment, the aminoglycoside agent is Lividomycin. In another preferred embodiment, the aminoglycoside agent is Gentamycin. In another preferred embodiment, the aminoglycoside is G418. It should be understood that particular aminoglycosides recited herein by name are also intended to include pharmaceutically acceptable salts thereof, such as sulfates thereof (e.g., gentamycin sulfate).

Aminoglycosides have been used both in vitro and in vivo for antibiotic purposes and, in certain limited situations, to suppress naturally-occurring stop codon mutations. Thus dosages and routes of administration of aminoglycosides that have been used to suppress naturally-occurring stop codon mutations also can be used to suppress the deliberate mutations of the invention. For example, a non-limiting dosage range of aminoglycoside for use with mammalian cells in culture is 0.05 mg/ml to 1 mg/ml, preferably 0.2 mg/ml. Aminoglycosides are commercially available (e.g., from Sigma Chemical Company) and can be added to culture medium in an aqueous solution. For in vivo treatment, pharmacokinetic data generated in the dog (Morris, T. H. (1995) Lab. Animal 29:16-36) can be used to calculate the allometric dose equivalent for other species of subjects (e.g., humans). For example, for mice an effective dosage for gentamycin sulfate has been found to be 17 mg/kg (Barton-Davis, E. R. (1999) J. Clin. Invest. 104:375-381). The aminoglycoside can be delivered by injection in vivo, such as by subcutaneous injection. Sustained treatment with the aminoglycoside may be necessary in vivo to maintain sustained expression of the gene of interest. For example, in one embodiment, the aminoglycoside is administered by subcutaneous injection at 100%, 200% and 400% of the calculated dose equivalents (based on Morris, T. H. supra) once per day for 14 days. In another embodiment, the aminoglycoside is administered using an osmotic pump. The osmotic pump can be implanted under the skin of the subject. In one embodiment, the pump is loaded with appropriate drug concentrations for the subject to receive 50%, 100% or 200% of the calculated dosage for two weeks.

When the aminoglycoside is to be used in vivo in a subject, a particular aminoglycoside is chosen that is suitable for in vivo use (e.g., an aminoglycoside in which any potential side effects are not so severe as to preclude use in vivo). A preferred aminoglycoside for use in vivo is Gentamycin (gentamycin sulfate).

Preferred dosage ranges, and potential toxicity, of particular aminoglycosides can be determined using in vitro systems (e.g., cultured cells, see Examples) or animal model systems. Moreover, the dosage of aminoglycoside to be used either in vitro or in vivo may be adjusted over time to thereby adjust the level of expression of the desired gene.

B. Suppressor tRNAs

In another embodiment of the invention, wherein a missense mutation(s) has been introduced into the gene, the agent is a suppressor tRNA molecule. Transfer RNA (tRNA) molecules are a class of ribonucleic acids that bring the individual amino acid residues to the translation machinery for addition to the nascent polypeptide chain. Each amino acid has one or a few specific tRNA molecules that are able to recognize the codon corresponding to its particular amino acid. This is accomplished through a three codon “anti-codon” in the tRNA molecule. Other stereo-specific considerations throughout the rest of the tRNA molecule also contribute to codon specificity, and are determined by the sequence of ribonucleotides comprising the tRNA. As used herein, the term “suppressor tRNA” refers to a modified tRNA molecule that has been altered such that it recognizes a missense or termination codon as it's amino acid codon, and subsequently add its amino acid onto the nascent amino acid chain. In one embodiment, the suppressor tRNA is provided directly to the cell from without (i.e. into the culture medium). In a preferred embodiment, a gene that codes for the suppressor tRNA is provided using standard gene expression vectors and transfer methodology as described in herein in the foregoing sections.

Particular suppressor tRNAs, and their corresponding codon specificity, have been described in the art. See for example, PCT Publication WO 99/36519 and European Patent Application EP 499 059, which describe oligonucleotide sequences of suppressor tRNAs that, when introduced into cells, can suppress nissense mutations. For example, PCT Publication WO 99/36519 describes human suppressor tRNAs that suppress UAG, UGA or UAA stop codon mutations by inserting either serine or arginine at the stop codon position (i.e., tRNAs that are charged with either serine or arginine and that recognize, and thereby suppress, either UAG, UGA or UAA stop codon mutations). Thus, for example, a stop codon mutation can be introduced into a desired gene of the invention at a codon position that naturally encodes serine or arginine and then the introduced stop codon mutation can be suppressed using the appropriate suppressor tRNA that is charged with either serine or arginine and the recognizes the introduced stop codon mutation.

C. Ribozyme Inhibitors

In another embodiment, wherein a mutation(s) that creates a ribozyme cleavage site has been introduced into the gene, the agent used to suppress the mutation is a ribozyme inhibitor. As used herein, the term “ribozyme inhibitor” refers to a molecule, including organic chemicals, peptides and nucleic acids, that can inhibit one or more particular ribozymes. A ribozyme inhibitor of the invention may inhibit the activity of the ribozyme either directly, e.g., by directly binding the ribozyme or by influencing the interaction of the ribozyme with its substrate RNA, or indirectly, e.g., by influencing the expression of the ribozyme. Preferred ribozyme inhibitor of the invention are aminoglycosides, which have been shown to be capable of inhibiting various ribozymes (see e.g., Walter, F. et al. (1999) Curr. Opin. Chem. Biol. 3:694-704; von Ahsen, U. et al. (1991) Nature 353:368-370; Rogers, J. et al. (1996) J. Mol. Biol. 259:916-925; Tor, Y. et al. (1998) Chem. Biol. 5:R277-283). Types of aminoglycosides that can be used to inhibit ribozyme activity, as well as dosages, routes of delivery and the like, include those described above in subsection IIA. In certain instances, the inhibitory ability of the aminoglycoside may require the presence of metal ions. For example, the ability of neomycin C to inhibit the activity of the hammerhead ribozyme has been shown to depend on the presence of metal ions (see Earnshaw, P. J. and Gait, M. J. (1998) Nucl. Acids Res. 26:5551-5561).

Alternatively, another type of ribozyme inhibitor is one that inhibits the expression of the ribozyme in the host cell of interest. A ribozyme can be introduced into the host cell in a vector in which transcriptional expression of the ribozyme is controlled by one or more regulatable elements. Thus, suppression of the mutation in the desired gene that creates a ribozyme cleavage site is achieved by use of an agent that inhibits the transcriptional expression of the ribozyme in the host cells. Regulatable (e.g., inducible) transcriptionally regulatory systems that can be used to control expression of the ribozyme are well known in the art (e.g., systems in which transcriptional expression is regulated by heavy metal ions, heat shock, hormones, tetracycline or rapamycin).

III. Uses of the Invention

The invention is widely applicable to a variety of situations where it is desirable to be able to turn gene expression on and off, or regulate the level of gene expression, in a rapid, efficient and controlled manner without causing pleiotropic effects or cytotoxicity. Thus, the system of the invention has widespread applicability to the study of cellular development and differentiation in eukaryotic cells, plants and animals. Additionally, the system can be used to regulate the expression of site-specific recombinases, such as CRE or FLP. Other applications of the regulatory system of the invention include:

A. Gene Therapy

The invention may be particularly useful for gene therapy purposes, in treatments for either genetic or acquired diseases. The general approach of gene therapy involves the introduction of nucleic acid into cells such that one or more gene products encoded by the introduced genetic material are produced in the cells to restore or enhance a functional activity. For reviews on gene therapy approaches see Anderson, W. F. (1992) Science 256:808-813; Miller, A.D. (1992) Nature 357:455-460; Friedmann, T. (1989) Science 244:1275-1281; and Cournoyer, D., et al. (1990) Curr. Opin. Biotech. 1:196-208. However, current gene therapy vectors typically utilize constitutive regulatory elements which are responsive to endogenous transcriptions factors. These vector systems do not allow for the ability to modulate the level of gene expression in a subject. In contrast, the inducible regulatory system of the invention provides this ability.

In addition to genes of interest already discussed herein (e.g., in subsection IC), genes of particular interest to be expressed in cells of a subject for treatment of genetic or acquired diseases include those encoding adenosine deaminase, β-globin, LDL receptor, anti-angiogenesis factors, glucocerebrosidase, β-glucouronidase, α1-antitrypsin, phenylalanine hydroxylase, tyrosine hydroxylase, ornithine transcarbamylase, arginosuccinate synthetase, UDP-glucuronysyl transferase, apoA1, TNF, soluble TNF receptor, interleukins (e.g., IL-2), interferons (e.g., α- or γ-IFN) and other cytokines and growth factors. Cells types which can be modified for gene therapy purposes include hematopoietic stem cells, myoblasts, hepatocytes, lymphocytes, skin epithelium, cardiomyocytes, vascular smooth muscle cells, endothelial cells, neurons and airway epithelium. For further descriptions of cell types, genes and methods for gene therapy see e.g., Wilson, J.M et al. (1 988) Proc. Natl. Acad. Sci. USA 85:3014-3018; Armentano, D. et al. (1990) Proc. Natl. Acad. Sci. USA 87:6141-6145; Wolff, J.A. et al. (1990) Science 247:1465-1468; Chowdhury, J.R. et al. (1991) Science 254:1802-1805; Ferry, N. et al. (1991) Proc. Natl. Acad. Sci. USA 88:8377-8381; Wilson, J. M. et al. (1992) J. Biol. Chem. 267:963-967; Quantin, B. et al. (1992) Proc. Natl. Acad. Sci. USA 89:2581-2584; Dai, Y. et al. (1992) Proc. Natl. Acad. Sci. USA 89:10892-10895; van Beusechem, V.W. et al. (1992) Proc. Natl. Acad. Sci. USA 89:7640-7644; Rosenfeld, M. A. et al. (1992) Cell 68:143-155; Kay, M. A. et al. (1992) Human Gene Therapy 3:641-647; Cristiano, R.J. et al. (1993) Proc. Natl. Acad. Sci. USA 90:2122-2126; Hwu, P. et al (993) J. Immunol. 150:4104-4115; and Herz, J. and Gerard, R. D. (1993) Proc. Natl. Acad. Sci. USA 90:2812-2816.

Gene therapy applications of particular interest in cancer treatment include overexpression of a cytokine gene (e.g., TNF-α) in tumor infiltrating lymphocytes or ectopic expression of cytokines in tumor cells to induce an anti-tumor immune response at the tumor site), expression of an enzyme in tumor cells which can convert a non-toxic agent into a toxic agent, expression of tumor specific antigens to induce an anti-tumor immune response, expression of tumor suppressor genes (e.g., p53 or Rb) in tumor cells, expression of a multidrug resistance gene (e.g., MDR1 and/or MRP) in bone marrow cells to protect them from the toxicity of chemotherapy.

Gene therapy applications of particular interest in treatment of viral diseases include expression of trans-dominant negative viral transactivation proteins, such as trans-dominant negative tat and rev mutants for HIV or trans-dominant ICp4 mutants for HSV (see e.g., Balboni, P. G. et al. (1993) J. Med. Virol. 41:289-295; Liem, S. E. et al. (1993) Hum. Gene Ther. 4:625-634; Malim, M.H. et al. (1992) J. Exp. Med. 176:1197-1201; Daly, T. J. et al. (1993) Biochemistry 32:8945-8954; and Smith, C. A. et al. (1992) Virology 191:581-588), expression of trans-dominant negative envelope proteins, such as env mutants for HIV (see e.g., Steffy, K. R. et al. (1993) J. Virol. 67:1854-1859), intracellular expression of antibodies, or fragments thereof, directed to viral products (“internal immunization”, see e.g., Marasco, W. A. et al. (1993) Proc. Natl. Acad. Sci. USA 90:7889-7893) and expression of soluble viral receptors, such as soluble CD4. Additionally, the system of the invention can be used to conditionally express a suicide gene in cells, thereby allowing for elimination of the cells after they have served an intended function. For example, cells used for vaccination can be eliminated in a subject after an immune response has been generated the subject by inducing expression of a suicide gene in the cells by administering an agent of the invention (e.g., an aminoglycoside) to the subject.

Gene therapy applications that may particularly benefit from the ability to regulate gene expression in an inducible manner in accordance with the methods of the invention include the following non-limiting examples:

Rheumatoid arthritis—genes which encode gene products that inhibit the production of inflammatory cytokines (e.g., TNF, IL-1 and IL-12). can be expressed in subjects. Examples of such inhibitors include soluble forms of a receptor for the cytokine. Additionally or alternatively, the cytokines IL-10 and/or IL-4 (which stimulate a protective Th2-type response) can be expressed. Moreover, a glucocorticomimetic receptor (GCMR) can be expressed.

Hypopituitarism—the gene for human growth hormone can be expressed in such subjects only in early childhood, when gene expression is necessary, until normal stature is achieved, at which time gene expression can be downregulated.

Wound healing/Tissue regeneration—Factors (e.g., growth factors, angiogenic factors, etc.) necessary for the healing process can be expressed only when needed and then downregulated. Anti-Cancer Treatments—Expression of gene products useful in anti-cancer treatment can be limited to a therapeutic phase until retardation of tumor growth is achieved, at which time expression of the gene product can be downregulated. Possible systemic anti-cancer treatments include use of tumor infiltrating lymphocytes which express immunostimulatory molecules (e.g., IL-2, IL-12 and the like), angiogenesis inhibitors (PF4, IL-12, etc.), Her-regulin, Leukoregulin (see PCT Publication No. WO 85/04662), and growth factors for bone marrow support therapy, such as G-CSF, GM-CSF and M-CSF. Regarding the latter, use of the regulatory system of the invention to express factors for bone marrow support therapy allows for simplified therapeutic switching at regular intervals from chemotherapy to bone marrow support therapy (similarly, such an approach can also be applied to AIDS treatment, e.g., simplified switching from anti-viral treatments to bone marrow support treatment). Furthermore, controlled local targeting of anti-cancer treatments are also possible. For example, expression of a suicide gene by a regulator of the invention, wherein the regulator itself is controlled by, for example, a tumor-specific promoter or a radiation-induced promoter.

In another embodiment, the regulatory system of the invention is used to express angiogenesis inhibitor(s) from within a tumor via a transgene regulated by the system of the invention. Expression of angiogenesis inhibitors in this manner may be more efficient than systemic administration of the inhibitor and would avoid any deleterious side effects that might accompany systemic administration. In particular, restricting angiogenesis inhibitor expression to within tumors could be particularly useful in treating cancer in children still undergoing angiogenesis associated with normal cell growth.

In another embodiment, high level regulated expression of cytokines may represent a method for focusing a patients own immune response on tumor cells. Tumor cells can be transduced to express chemoattractant and growth promoting cytokines important in increasing an individual's natural immune response. Because the highest concentrations of cytokines will be in the proximity of the tumor, the likelihood of eliciting an immunological response to tumor antigens is increased. A potential problem with this type of therapy is that those tumor cells producing the cytokines will also be targets of the immune response and therefor the source of the cytokines will be eliminated before eradication of all tumor cells can be certain. To combat this, expression of viral proteins known to mask infected cells from the immune system can be placed under regulation, along with the cytokine gene(s), in the same cells. One such protein is the E19 protein from adenovirus (see e.g., Cox, Science 247:715). This protein prevents transport of class I HLA antigens to the surface of the cell and hence prevents recognition and lysis of the cell by the host's cytotoxic T cells. Accordingly, regulated expression of E19 in tumor cells could shield cytokine producer cells from cytotoxic T cells during the onset of an immune response provoked by cytokine expression. After a sufficient period of time has elapsed to eradicate all tumor cells but those expressing E19, E19 expression can be turned off, causing these cells then to fall victim to the provoked anti-tumor immune response.

Benign prostatic hypertrophy—Similar to the above, a suicide gene can be regulated by a regulator of the invention, wherein the regulator itself is controlled by, for example, a prostate-specific promoter.

The ability to express a suicide gene (e.g., an apoptosis gene, TK gene, etc) in a controlled manner using the regulatory system of the invention adds to the general safety and usefulness of the system. For example, at the end of a desired therapy, expression of a suicide gene can be triggered to eliminate cells carrying the gene therapy vector, such as cells in a bioinert implant, cells that have disseminated beyond the intended original location, etc. Moreover, if a transplant becomes tumorous or has side effects, the cells can be rapidly eliminated by induction of the suicide gene.

The regulatory system of the invention further offers the ability to establish a therapeutically relevant expression level for a gene product of interest in a subject, in contrast to unregulated constitutive expression which offers no flexibility in the level of gene product expression that can be achieved. A physiologically relevant level of gene product expression can be established based on the particular medical need of the subject, e.g., based on laboratory tests that monitor relevant gene product levels (using methods as described above). Therapeutically relevant gene products which can be expressed at a desired level at a desired time include: Factor XIII and IX in hemophiliacs (e.g., expression can be elevated during times of risk of injury, such as during sports); insulin or amylin in diabetics (as needed, depending on the state of disease in the subject, diet, etc.); erythropoietin to treat erythrocytopenia (as needed, e.g., at end-stage renal failure); low-density lipoprotein receptor (LDLr) or very low-density lipoprotein receptor (VLDLr) for artherosclerosis or gene therapy in liver (e.g, using ex vivo implants). Applications to treatment of central nervous system disorders are also encompassed. For example, in Alzheimer's disease, “fine tuned” expression of choline acetyl transferase (ChAT) to restore acetylcholine levels, neurotrophic factors (e.g., NGF, BDNGF and the like) and/or complement inhibitors (e.g., sCR1, sMCP, sDAF, sCD59 etc.) can be accomplished. Such gene products can be provided, for example, by transplanted cells expressing the gene products in a regulated manner using the system of the invention. Moreover, Parkinson's disease can be treated by “fine tuned” expression of tyrosine hydroxylase (TH) to increase levodopa and dopamine levels.

B. Production of Proteins in vitro

Large scale production of a protein of interest can be accomplished using cultured cells in vitro and the regulatory methods of the invention. Accordingly, the invention provides a production process for producing and isolating a protein of interest in eukaryotic cells (e.g., mammalian cells, yeast cells). Once protein expression has been induced using the regulatory system of the invention (as described infra), standard protein purification techniques can be used to isolate the protein of interest from the medium or from the harvested cells.

C. Production of Proteins in vivo

The invention also provides for production of a protein of interest in animals, such as in transgenic farm animals. Advances in transgenic technology have made it possible to produce transgenic livestock, such as cattle, goats, pigs and sheep (reviewed in Wall, R. J. et al. (1992) J. Cell. Biochem. 49:113-120; and Clark, A. J. et al. (1987) Trends in Biotechnology 5:20-24). Accordingly, transgenic livestock carrying in their genome the components of the inducible regulatory system of the invention can be constructed and gene expression can be modulated using an agent of the invention. Protein production can be targeted to a particular tissue by linking the gene of interest to an appropriate tissue-specific regulatory element(s) which limits expression of the gene to certain cells. For example, a mammary gland-specific regulatory element, such as the milk whey promoter (U.S. Pat. No. 4,873,316 and European Application Publication No. 264,166), can be linked to the gene of interest to limit expression of the transactivator to mammary tissue. Thus, in the presence of an agent of the invention (e.g., aminoglycoside), the gene product encoded by the gene of interest will be produced in the mammary tissue of the transgenic animal. The protein can be designed to be secreted into the milk of the transgenic animal, and if desired, the protein can then be isolated from the milk.

IV. Nucleic Acid Compositions of the Invention

Another aspect of the invention pertains to nucleic acid compositions for use in the regulatory methods of the invention. Such nucleic acid compositions comprise a gene of interest into which has been introduced a non-naturally occurring mutation that renders the gene of interest suitable for regulation in accordance with the methods of the invention.

For example, in one embodiment, the invention provides an isolated human erythropoietin gene comprising at least one stop codon mutation within the coding region of the gene. Preferred mutated Epo constructs of the invention include constructs in which the human Epo cDNA is mutated at nucleotide position 199, 208, 214, 307, 434, 437, 454 or 526, or in which the human Epo cDNA is mutated at a codon encoding amino acid position 6, 9, 11, 62, 85, 86, 91 or 115. More preferred constructs are those mutated at nucleotide position 199, 208 or 214, or at a codon encoding amino acid position 6, 9 or 11.

In other embodiments, the invention provides an isolated human insulin gene comprising at least one stop codon mutation within the coding region of the gene; an isolated human VEGF gene comprising at least one stop codon mutation within the coding region of the gene; an isolated human FGF gene comprising at least one stop codon mutation within the coding region of the gene; an isolated human HIF-1α gene comprising at least one stop codon mutation within the coding region of the gene; an isolated human Factor VIII gene comprising at least one stop codon mutation within the coding region of the gene; an isolated human Factor IX gene comprising at least one stop codon mutation within the coding region of the gene; an isolated human growth hormone gene comprising at least one stop codon mutation within the coding region of the gene; an isolated human endostatin gene comprising at least one stop codon mutation within the coding region of the gene; an isolated human angiostatin gene comprising at least one stop codon mutation within the coding region of the gene; and an isolated herpes simplex virus thymidine kinase gene comprising at least one stop codon mutation within the coding region of the gene. It should be understood the “at least one stop codon mutation” referred to for each of the afore-mentioned genes is not intended to include the naturally-occurring stop codon that exists at the translational termination site in the gene, but rather refers to a non-naturally occurring stop codon mutation that is introduced into the coding region of the gene.

In other embodiments, the invention provides the afore-mentioned isolated genes having at least one missense mutation within the coding region of the gene. In yet another embodiment, the invention provides the afore-mentioned isolated genes having at least one mutation that creates a ribozyme cleavage site introduced into the gene. In yet other embodiments, the invention provides isolated genes corresponding to any of the genes of therapeutic interest discussed in subsection III above, into which has been introduced at least one stop codon mutation, or at least one missense mutation or at least one mutation that creates a ribozyme cleavage site.

V. Kits of the Invention

Another aspect of the invention pertains to kits that contain components of the inducible expression system of the invention. Such a kit can be used to regulate expression of a gene in cultured cells or in tissues of a host organism. Such a kit can be further used to regulate expression of a desired gene in vitro (e.g., in cultured cells) or for therapeutic purposes (e.g., in vivo in a subject).

In one embodiment, the kit contains a desired gene, into which a mutation(s) of the invention has already been introduced, as described herein, together with an agent that suppresses the mutation(s) in a eukaryotic cell. In a preferred embodiment, the kit contains the mutated, desired gene wherein the gene is incorporated into an expression vector, as described herein. Preferred desired genes for inclusion in the kit include those described in subsection IV above. In a preferred embodiment, the kit contains a preparation of aminoglycoside as the suppressor agent. Preferred aminoglycosides include Gentamycin, Hygromycin-B, Paromomycin, Tobramycin, Lividomycin and G418, and pharmaceutically acceptable salts thereof. In other embodiments, the kit contains suppressor tRNA as the suppressor agent, such as a gene for a suppressor tRNA, or a gene for suppressor tRNA contained in an appropriate vector for co-expression in host cells with the desired gene. In yet another embodiment, the kit contains a ribozyme inhibitor as the suppressor agent.

In another embodiment, the kit can further contain material and reagents needed to transfer the nucleic acids of the invention into host cells, as described previously herein (e.g. transfection reagents). In a further embodiment, the kit includes cells that can be cultured and used as host cells of the invention. In another embodiment, the kit includes cultured cells into which a mutated, desired gene of the invention has been transferred.

The present invention is further illustrated by the following examples, which should not be construed as limiting in any way. The contents of all cited references, including literature references, issued patents, and published patent applications, as cited throughout this application are hereby expressly incorporated by reference.

### EXAMPLE 1.

### Introduction of Stop Codon Mutations into the Human Erythropoietin Gene

In this example, PCR-based mutagenesis was used to introduce three different stop codon mutations into the coding region of the human erythropoietin gene.

The human erythropoietin gene was PCR amplified from the plasmid pAdEF1hEpo (Tripathy et al 1994, PNAS 91:11557-11561) using the primers:

5′hEpo 5′-GAGACTGAAGTTAGGCCAGCTTGG-3′ (SEQ ID NO:1), and

3′hEpo 5′-CATCTAGATCCGTCTGGGGACAGG-3′ (SEQ ID NO:2).

Three nested pairs of complementary primers specific for the coding region of hEpo were synthesized such that one nucleotide is mismatched (indicated in bold). These mismatches created one of three different stop codon mutations in the hEpo gene. The primers used to accomplish this were:

5′hEpo.437 5′-GGATGGAGGTCGGGCAGTAGGCCGTAGAAGTCTGGCAGGG-3′(SEQ ID NO:3) and

3′hEpo.437 5′-CCCTGCCAGACTTCTACGGCCTACTGCCCGACCTCCATCC-3′(SEQ ID NO:4), which created a stop mutation at codon 86,

5′hEpo.454 5′-GCAGCAGGCCGTAGAAGTCTGACAGGGCCTGGCCCTGCTG-3′(SEQ ID NO:5) and

3′hEpo.454 5′-CAGCAGGGCCAGGCCCTGTCAGACTTCTACGGCCTGCTGC-3′(SEQ ID NO:6), which created a stop mutation at codon 91,

5′hEpo.526 5′-GTCAACTCTTCCCAGCCGTGAGAGCCCCTGCAGCTGCATG-3′(SEQ ID NO:7), and

3′hEpo.526 5′-CATGCAGCTGCAGGGGCTCTCACGGCTGGGAAGAGTTGAC-3′(SEQ ID NO:8), which created a stop mutation at codon 115.

PCR reactions contained 100 ng of pAdEF1hEpo DNA template, 1 μg of one of the three pairs of nested primers, 25 mM of dATP, dCTP, dGTP, and dTTP, 1× Pfu polymerase buffer and Pfu polymerase. The thermal cycling for PCR reactions was as follows:

A) one denaturation step of 94° C. for 2 minutes,

B) step-down PCR:

a) 94° C. for 5 seconds and 72° C. for 2 minutes (5 cycles)

b) 94° C. for 5 seconds and 70° C. for 2 minutes (5 cycles)

c) 94° C. for 5 seconds and 68° C. for 2 minutes (20 cycles)

C) one elongation step of 72° C. for 10 minutes

D) storage of reaction at 4° C.

Reactions were run on a 1.0% agarose gel to remove excess primers from DNA, and subsequently purified of contaminating agarose using Qiaquick gel isolation kit (Qiagen). Amplified product overhangs were filled in using T4 DNA polymerase and blunt-end ligated into the MCS (multiple cloning site) of pVR1012 the EcoRV site. Within the pVR1012 vector, expression of the introduced Epo sequence is controlled by a CMV promoter and enhancer. Ligation products were introduced into competent bacteria, and various clones picked and cultured in a volume of liquid media. Plasmid DNA from these bacterial clone cultures was isolated, checked for correct size by restriction digest and sequenced to confirm the orientation and integrity of the hEpo coding region and newly-introduced stop codon mutations. Three clones were selected, each containing one of the three intended stop codon mutations in the hEpo gene. DNA was quantified based on spectrophotometric absorbance.

### EXAMPLE 2.

### Regulation of hEpo Expression in Myoblasts with Aminoglycosides

In this example, three different mutated human Epo genes (prepared as described in Example 1) were individually transfected into myoblasts and expression of the genes was subsequently regulated by application of two different aminoglycosides.

The C2C12 mouse myoblast cell line (ATCC) was maintained in DMEM, 10% fetal bovine serum, 1% penicillin/streptomycin and 1% glutamine.

C2C 12 cells were plated at 5×10⁵ cells/cm² on the afternoon before transfection. 15 μg of wildtype or mutated hEpo plasmid DNA was mixed with 50 μl of Lipofectamine (Gibco) and 2 mls of Opti-MEM (Gibco) and incubated at room temperature for 15 minutes for DNA/liposome complexes to form. Cells were washed twice with 5 mils of Opti-MEM. Two ml of Opti-MEM and the DNA/liposome complexes were added and incubated at 37° C. in 5% CO₂ for 5 hours. Eight ml of growth medium (DMEM+10% FBS) was added to cells and incubated overnight at 37° C.

The following day, 10 mls of fresh growth medium was added to the cells with or without 0.2 mg/ml of one of two aminoglycosides: G418 (Sigma) or gentamycin sulfate (Sigma). Cells were allowed to incubate for an additional 72 hours and a sample of the supernatant was taken for transgene analysis at 24, 48, and 72 hours.

Supernatants from transfected cells were analyzed for hEpo expression using the Quantikine IV human Epo ELISA kit (R&D Systems). Samples were diluted within the linear range of the assay using specimen diluent and assayed using a microplate reader (Molecular Devices) at 450 nm.

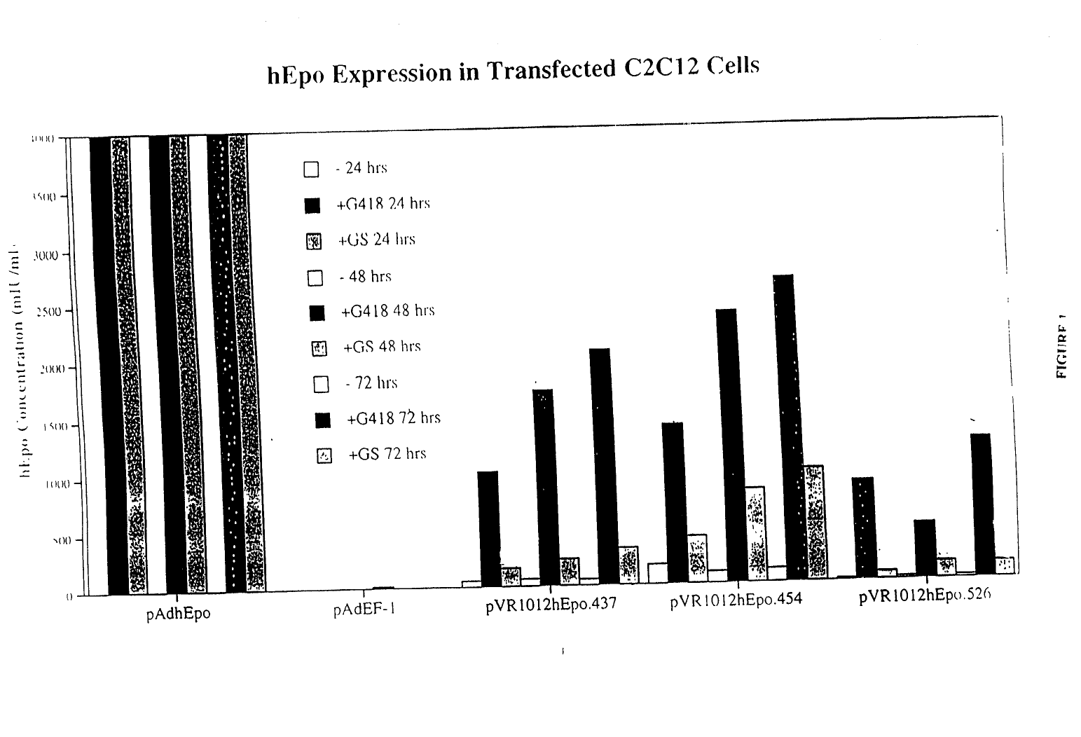

Results are summarized below in Table 1 and illustrated in the bar graph of FIG. 1.

TABLE 1
Comparison of results of conditional hEpo expression at 24, 48, and 72 hours in the
presence of the aminoglycosides G418 or gentamycin.

| | | 24 hrs | 24 hrs | | 48 hrs | 48 hrs | | 72 hrs | 72 hrs |
|---|---|---|---|---|---|---|---|---|---|
| | 24 hrs | +G418 | +GS | 48 hrs | +G418 | +GS | 72 hrs | +G418 | +GS |
| pAdhEpo | 35580 | 35580 | 35580 | 136935 | 119488 | 125561 | 152417 | 145748 | 158431 |
| PAdEF-1 | 2.58 | 2.93 | 2.81 | 3.17 | 2.81 | 21.51 | 3.05 | 3.05 | 2.7 |
| Stop 86 | 56.41 | 1018.56 | 167.48 | 66.29 | 1721.21 | 239.69 | 56.88 | 2064.2 | 325.44 |
| Stop 91 | 176.21 | 1406.8 | 421.15 | 107.02 | 2376.22 | 828.01 | 122.5 | 2669.19 | 997.12 |
| Stop 115 | 14.96 | 882.79 | 72.21 | 20.68 | 496.93 | 156.32 | 24.97 | 1244.84 | 151.56 |

Cells incubated in the presence of G418 demonstrated a 20-50 fold induction of hEpo expression compared to cells that were not given aminoglycoside antibiotics (FIG. 1). The expression of hEpo increased over time for the duration of the experiment. Cells incubated in the presence of gentamycin sulfate also demonstrated induction of hEpo expression, although only a 6-8 fold induction of hEpo expression was observed. Thus, there was less read-through of the stop codon mutation with gentamycin sulfate (3%) than with G418 (10%) in vitro.

Interestingly, mutations of different amino acids within the transgene gave varying amounts of read-through efficiency. While p1012hEpo.454 (mutated at codon 91) (22 fold) and p1012hEpo.437 (mutated at codon 86) demonstrated higher overall hEpo expression levels of hEpo under permissive conditions (presence of aminoglycoside), p1012hEpo.526 (mutated at codon 115) gave the highest induction rate (52 fold) due to less background readthrough in the absence of aminoglycoside. In addition, it was noted that p1012hEpo.454 (mutated at codon 91) demonstrated the highest induction and expression levels with the antibiotic gentamycin sulfate. Thus, aminoglycoside antibiotics can induce read-through of nonsense mutations in vitro in a muscle cell line.

### EXAMPLE 3

### Inducibility of Additional Mutant hEpo Constructs

In this example, a series of additional mutated human Epo constructs were prepared, into which stop codon mutations were introduced at different codon positions from those described in Example 1. These constructs were introduced into C2C12 mouse myoblasts as described in Example 2 and the inducibility of hEpo activity from these constructs using either gentamycin sulfate (GS) or G418 was examined as described in Example 2. The constructs were prepared by standard PCR mutagenesis, as described in Example 1, and introduced a stop codon mutation into the human Epo sequence at either nucleotide position 307 (pVR1012hEpo.307), position 434 (pVR1012hEpo.434), position 208 (pVR1012hEpo.208), position 214 (pVR1012hEpo.214) or position 199 (pVR1012hEpo. 199).

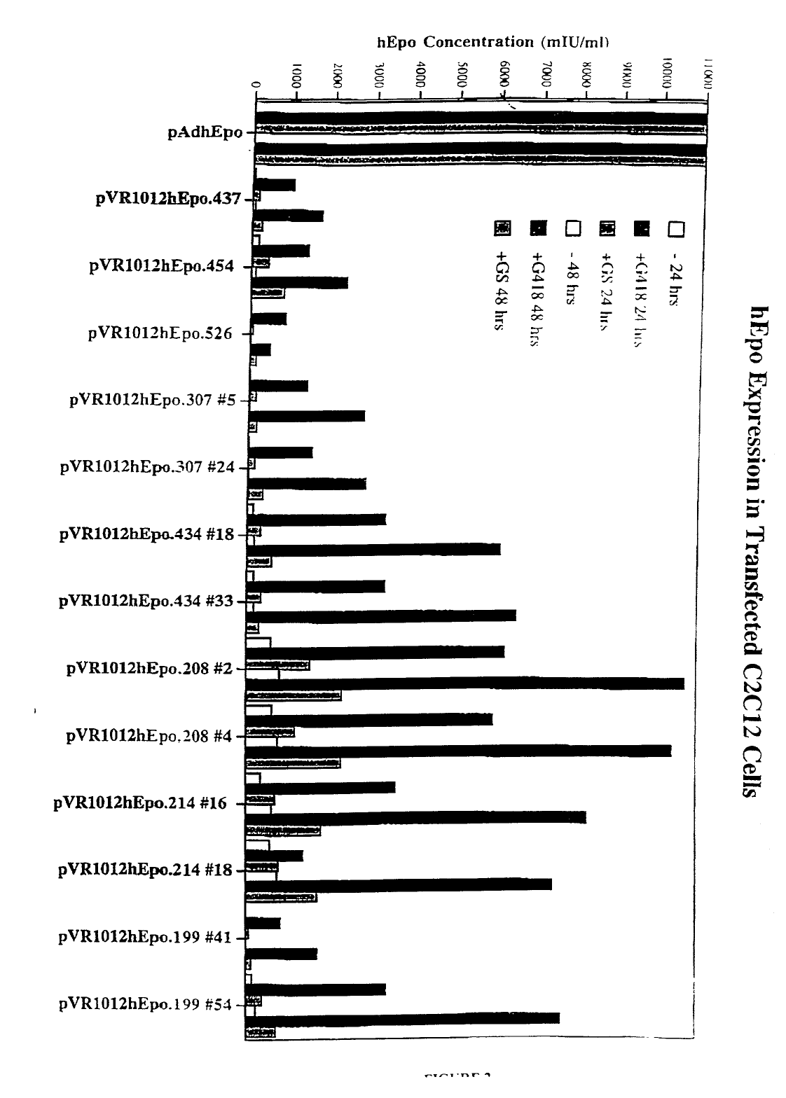

The results of the transfected cell experiments for these constructs (two clones for each construct) are shown in FIG. 2, which shows the concentration of hEpo expressed by the cells either in the absence of aminoglycoside treatment or in the presence of either gentamycin sulfate (GS) or G418, after 24 or 48 hours treatment. Data for constructs with mutations at either nucleotide position 437 (pVR1012hEpo.437), position 454 (pVR1012hEpo.454) or position 526 (pVR1012hEpo.526), prepared as described in Example 1, are included for comparison purposes. The results demonstrate that all of the additional constructs also exhibit inducibility by aminoglycoside treatment and that constructs having a stop codon introduced at either nucleotide position 208, 214 or 199 (pVR1012hEpo.208, pVR1012hEpo.214 or pVR1012hEpo. 199, respectively) are particularly responsive to aminoglycoside induction.

### EXAMPLE 4

### Functional Activity of hEpo Induced by Aminoglycoside Treatment

In this example, the functional activity of human Epo induced upon aminoglycoside treatment of C2C12 cells transfected with one of several different mutant hEpo constructs described in Examples 1 and 3 was assessed using UT7 cells. UT7 cells are dependent on Epo for growth. This assay measures the proliferation of UT7 cells in the presence of culture supernatants from the transfected muscle cells and compares it to proliferation of the same cells in the presence of known concentrations of recombinant human Epo. It therefore measures the biological activity of the Epo produced by the transfected cells. The results are shown in FIG. 4, which depicts the results for cells transfected with constructs having a stop codon mutation at either nucleotide position 437 (pVR1012hEpo.437), position 454 (pVR1012hEpo.454), position 199 (pVR1012hEpo.199), position 208 (pVR1012hEpo.208 ) or position 214 (pVR1012hEpo.214). The results demonstrate that for each of these constructs, functional hEpo activity was induced upon treatment with the aminoglycoside G418.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of regulating expression of a gene in a eukaryotic cell, which method comprises:
introducing at least one mutation into a transcribed region of the gene such that translation of the gene terminates at or is altered at the at least one mutation and a functional gene product encoded by the gene is not produced in the cell; and
contacting the eukaryotic cell with an agent that suppresses the at least one mutation of the gene such that a functional gene product of the gene is produced in the eukaryotic cell, thereby regulating expression of the gene in the eukaryotic cell.

2. The method of claim 1, wherein the at least one mutation in the gene creates at least one stop codon in a coding region of the gene.

3. The method of claim 2, wherein the agent is an aminoglycoside.

4. The method of claim 1, wherein the at least one mutation in the gene creates at least one missense mutation in a coding region of the gene.

5. The method of claim 4, wherein the agent is a suppressor tRNA.

6. The method of claim 1, wherein the mutation creates a ribozyme cleavage site that is cleaved by a ribozyme in the eukaryotic cell.

7. The method of claim 6, wherein the agent is an inhibitor of the ribozyme that cleaves the ribozyme cleavage site.

8. The method of claim 1, wherein the gene is a eukaryotic gene.

9. The method of claim 1, wherein the gene is a viral gene.

10. The method of claim 1, wherein the gene is a gene of therapeutic interest.

11. A method of regulating expression of a gene in a eukaryotic cell, which method comprises:
introducing at least one stop codon mutation into a coding region of the gene such that translation of the coding region terminates at the at least one stop codon mutation and a functional gene product encoded by the gene is not produced in the cell, and
contacting the eukaryotic cell with an aminoglycoside such that the functional product of the gene is produced in the eukaryotic cell, thereby regulating expression of the gene in the eukaryotic cell.

12. The method of claim 11, wherein the gene is a eukaryotic gene.

13. The method of claim 12, wherein the eukaryotic gene is selected from the group consisting of: erythropoietin, insulin, vascular endothelial cell growth factors (VEGFs), fibroblast growth factors (FGFs), Hypoxia-Inducing Factor-1α (HIF-1α), Factor VIII, Factor IX, Growth Hormone, endostatin, angiostatin and Herpes Simplex Virus Thymidine Kinase.

14. The method of claim 11, wherein the gene is a viral gene.

15. The method of claim 11, wherein the gene is a gene of therapeutic interest.

16. The method of claim 11, wherein the aminoglycoside is selected from the group consisting of: Hygromycin-B, Gentamycin, Paromomycin, Tobramycin, and Lividomycin and G418.

17. The method of claim 11, wherein the gene is an exogenous gene that has been delivered into the eukaryotic cell, using a eukaryotic expression vector, after introducing the at least one stop codon mutation into the coding region of the gene.

18. The method of claim 17, wherein the eukaryotic expression vector is a retroviral vector or a lentiviral vector.

19. The method of claim 17, wherein the eukaryotic expression vector is an adenoviral vector or an adeno-associated viral vector.

20. The method of claim 17, wherein the gene is introduced into cultured cells in vitro.

21. The method of claim 20, wherein the gene is introduced into the cultured cells using a method selected from the group consisting of: transfection, lipofection, calcium phosphate precipitation, electroporation and viral infection.

22. The method of claim 20, which further comprises administering the cultured cells to a subject after introducing the gene into the cultured cells.

23. The method of claim 11, which further comprises introducing the gene into cells of a subject in vivo after introducing the at least one stop codon mutation into the coding region of the gene in vitro.

24. The method of claim 20, wherein the cultured cells are selected from the group consisting of hematopoietic stem cells, myoblasts, hepatocytes, lymphocytes, skin epithelium, cardiomyocytes, vascular smooth muscle cells, endothelial cells, neurons and airway epithelium.

25. A method of regulating expression of an erythropoietin gene in a eukaryotic cell, which method comprises:
introducing at least one stop codon mutation into a coding region of the erythropoietin gene, such that translation of the coding region terminates at the at least one stop codon mutation and a functional erythropoietin gene product is not produced in the cell, and
contacting the eukaryotic cell with an aminoglycoside such that the at least one stop codon mutation is suppressed and a functional erythropoietin gene product is produced in the eukaryotic cell, thereby regulating expression of the erythropoietin gene in the eukaryotic cell.

26. The method of claim 25, wherein the aminoglycoside is Gentamycin.

27. The method of claim 25, wherein the erythropoietin gene is a human erythropoietin gene that is mutated at a codon encoding amino acid position 6, 9, 11, 62, 85, 86, 91 or 115 of human erythropoietin.

28. The method of claim 25, wherein the erythropoietin gene is a human erythropoietin gene that is mutated at a codon encoding amino acid position 6, 9 or 11 of human erythropoietin.

29. A method of regulating expression of a gene in a eukaryotic cell, which method comprises:
introducing at least one stop codon mutation into a coding region of the gene, such that translation of the coding region terminates at the at least one stop codon mutation and a functional gene product is not produced in the cell, wherein the gene is selected from the group consisting of insulin, vascular endothelial cell growth factors (VEGFs), fibroblast growth factors (FGFs), hypoxia-inducing factor-1α (HIF-1α), Factor VIII, Factor IX, growth hormone, endostatin, angiostatin and Herpes Simplex Virus Thymidine Kinase; and
contacting the eukaryotic cell with Gentamycin such that the at least one stop codon mutation is suppressed and a functional gene product is produced in the eukaryotic cell, thereby regulating expression of the gene in the eukaryotic cell.

30. An isolated nucleic acid molecule comprising a human erythropoietin gene comprising at least one stop codon mutation in a coding region of the gene.

31. The isolated nucleic acid molecule of claim 30, wherein the human erythropoietin gene comprises a stop codon mutation at a codon encoding amino acid position 6, 9, 11, 62, 85, 86, 91 or 115 of human erythropoietin.

32. The isolated nucleic acid molecule of claim 30, wherein the human erythropoietin gene comprises a stop codon mutation at a codon encoding amino acid position 6, 9 or 11 of human erythropoietin.

33. A eukaryotic expression vector comprising the nucleic acid molecule of claim 30.

34. An isolated nucleic acid molecule comprising a gene comprising at least one stop codon mutation in a coding region of the gene, wherein the gene is selected from the group consisting of human insulin, human vascular endothelial cell growth factors (VEGFs), human fibroblast growth factors (FGFs), human hypoxia-induced factor-1α (HIF-1α), human Factor VIII, human Factor IX, human growth hormone (HGH), human endostatin, human angiostatin and herpes simplex virus thymidine kinase.

35. A eukaryotic expression vector comprising the nucleic acid molecule of claim 34.

36. A kit for regulating expression of a gene, said kit comprising an isolated gene comprising at least one stop codon mutation within a coding region of the gene and an aminoglycoside for suppression of the at least one stop codon mutation in the gene.

37. The kit of claim 36, wherein the gene is a human erythropoietin gene and the aminoglycoside is Gentamycin.


SEQUENCE LISTING (8 sequences)

SEQ ID NO: 1 - DNA, 24 bp, Homo sapiens
gagactgaagttaggccagcttgg

SEQ ID NO: 2 - DNA, 24 bp, Homo sapiens
catctagatccgtctggggacagg

SEQ ID NO: 3 - DNA, 40 bp, Homo sapiens
ggatggaggtcgggcagtaggccgtagaagtctggcaggg

SEQ ID NO: 4 - DNA, 40 bp, Homo sapiens
ccctgccagacttctacggcctactgcccgacctccatcc

SEQ ID NO: 5 - DNA, 40 bp, Homo sapiens
gcagcaggccgtagaagtctgacagggcctggccctgctg

SEQ ID NO: 6 - DNA, 40 bp, Homo sapiens
cagcagggccaggccctgtcagacttctacggcctgctgc

SEQ ID NO: 7 - DNA, 40 bp, Homo sapiens
gtcaactcttcccagccgtgagagcccctgcagctgcatg

SEQ ID NO: 8 - DNA, 40 bp, Homo sapiens
catgcagctgcaggggctctcacggctgggaagagttgac